SEQUENCE LISTING (36 sequences)

SEQ ID NO: 1 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 444 aa, Homo sapiens
QVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 118 aa, Homo sapiens
QVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSS

SEQ ID NO: 4 - protein, 10 aa, Homo sapiens
GFTFSSHGMH

SEQ ID NO: 5 - protein, 15 aa, Homo sapiens
VIWYDGRNKYYADSV

SEQ ID NO: 6 - protein, 9 aa, Homo sapiens
GGHFGPFDY

SEQ ID NO: 7 - DNA, 708 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcagcttcttagcctggtaccagcagagacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 215 aa, Homo sapiens
EIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 108 aa, Homo sapiens
EIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIK

SEQ ID NO: 10 - protein, 12 aa, Homo sapiens
RASQSISSSFLA

SEQ ID NO: 11 - protein, 7 aa, Homo sapiens
GASSRAT

SEQ ID NO: 12 - protein, 9 aa, Homo sapiens
QQYGTSPWT

SEQ ID NO: 13 - DNA, 1335 bp, Homo sapiens
caggtgcagctggtggagtctgggggaggcgtggtccagcctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 444 aa, Homo sapiens
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 118 aa, Homo sapiens
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSS

SEQ ID NO: 16 - protein, 10 aa, Homo sapiens
GFTFSSHGIH

SEQ ID NO: 17 - protein, 15 aa, Homo sapiens
VIWYDGRNKDYADSV

SEQ ID NO: 18 - protein, 9 aa, Homo sapiens
VAPLGPLDY

SEQ ID NO: 19 - DNA, 645 bp, Homo sapiens
gaaattgtgttgacgcagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 107 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIK

SEQ ID NO: 22 - protein, 11 aa, Homo sapiens
RASQSVSSYLA

SEQ ID NO: 23 - protein, 7 aa, Homo sapiens
GASSRAT

SEQ ID NO: 24 - protein, 9 aa, Homo sapiens
QQYGRSPFT

SEQ ID NO: 25 - DNA, 1413 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 125 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 28 - protein, 10 aa, Homo sapiens
GFTFSSYGMH

SEQ ID NO: 29 - protein, 15 aa, Homo sapiens
VIWYDGSNKYYADSV

SEQ ID NO: 30 - protein, 16 aa, Homo sapiens
DPRGATLYYYYYGMDV

SEQ ID NO: 31 - DNA, 714 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 32 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 107 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 34 - protein, 11 aa, Homo sapiens
RASQSINSYLD

SEQ ID NO: 35 - protein, 7 aa, Homo sapiens
AASSLQS

SEQ ID NO: 36 - protein, 9 aa, Homo sapiens
QQYYSTPFT

(19) United States
(12) Patent Application Publication
(10) Pub. No.: US 2008/0279865 A1
(43) Pub. Date: Nov. 13, 2008

(54) Therapy of Prostate Cancer With Ctla-4 Antibodies and Hormonal Therapy

(75) Inventor: Jesus Gomez-Navarro, Mystic, CT (US)

Correspondence Address: PFIZER INC, 10555 SCIENCE CENTER DRIVE, SAN DIEGO, CA, 92121, US

(73) Assignee: Pfizer, Inc., Pfizer Products, Inc.

(21) Appl. No.: 11/817,390

(22) PCT Filed: Mar. 3, 2006
(86) PCT No.: PCT/US06/07650

§ 371 Date: Apr. 7, 2008

Related U.S. Application Data
(60) Provisional application No. 60/664,364, filed on Mar. 23, 2005.
(60) Provisional application No. 60/711,707, filed on Aug. 26, 2005.

(51) Int. Cl. A61K 39/395; A61P 35/00

(52) U.S. Cl. 424/172.1

(57) ABSTRACT

The invention relates to methods for treating prostate cancer comprising administration of an anti-CTLA4 antibody, or an antigen-binding portion thereof, particularly a human antibody to human CTLA4, e.g., antibody 3.1.1, 4.1.1, 4.8.1, 4.10.2, 4.13.1, 4.14.3, 6.1.1, ticilimumab (also known as 11.2.1), 11.6.1, 11.7.1, 12.3.1.1, 12.9.1.1, and ipilimumab (also known as MDX-010 and 10D1), in combination with hormonal therapy. Hormonal therapy agents include, inter alia, an anti-androgen (e.g., megestrol, cyproterone, flutamide, nilutamide, and bicalutamide), a GnRH antagonist (e.g., abarelix and histrelin), and a LH-RH agonist (e.g., leuprolide, goserelin, and buserelin). The invention relates to neoadjuvant therapy, adjuvant therapy, therapy for rising PSA, first-line therapy, second-line therapy, and third-line therapy of prostate cancer, whether localized or metastasized.

### BACKGROUND OF THE INVENTION

Due to the lack of effective treatments, prostate cancer is now the second most common cause of male cancer death. Approximately one-third of men who undergo radical prostatectomy for clinically localized prostate cancer will require treatment for recurrent prostate cancer within five years (Syed et al., Urol. Oncol. 21:235-243 (2003)). Patients that recur biochemically (i.e., those who have an increase in their prostate-specific antigen [PSA] level post surgery or post radiotherapy) increasingly receive hormone therapy (HT, also referred to herein as “androgen suppression therapy”, “androgen ablation therapy”, and “anti-androgen therapy”). Alternatively, hormone therapy may be delayed until clinical evidence of metastatic disease appears. Eventually, a majority of patients will become refractory to hormone therapy, and many will die due to cancer progressions. Thus, despite advances, including hormone therapy, there is a long-felt and unmet need for novel methods of treatment for prostate cancer.

Current antitumor agents act by a variety of mechanisms that inhibit cancer cell growth and division, ultimately destroying the malignant cell. However, because these cytotoxic agents are generally not selective for neoplastic cells, they destroy normal cells, disrupt physiologic functions, and are often associated with adverse effects. An alternative approach to cancer therapy is to target the immune system (“immunotherapy”) rather than the tumor itself so that the patient's own immune system attacks tumors while sparing non-tumor cells.

One cancer immunotherapy approach targets cytotoxic T lymphocyte-associated antigen 4 (CTLA4; CD152), which is a cell surface receptor expressed on activated T cells. Binding of CTLA4 to its natural ligands, B7.1 (CD80) and B7.2 (CD86), delivers a negative regulatory signal to T cells, and blocking this negative signal results in enhanced T cell immune function and antitumor activity in animal models (Thompson and Allison Immunity 7:445-450 (1997); McCoy and LeGros Immunol.& Cell Biol. 77:1-10 (1999)). Several studies have demonstrated that CTLA4 blockade using antibodies markedly enhances T cell-mediated killing of tumors and can induce antitumor immunity (Leach et al., Science 271:1734-1736 (1996); Kwon et al. Proc. Natl. Acad. Sci. USA 94:8099-8103 (1997); Kwon et al., Natl. Acad. Sci. USA 96:15074-15079 (1999)).

Anti-CTLA4 antibodies hold great promise in the treatment of cancer, but there is a long-felt need to develop novel immunotherapies to treat tumors with such antibodies while reducing the cytotoxic side effects of current chemotherapeutics. The present invention meets this need.

### SUMMARY OF THE INVENTION

The invention includes a method for the treatment of prostate cancer in a patient in need of such treatment. The method comprises administering to the patient a) an amount of a hormonal therapy agent and b) an amount of an antibody, or antigen-binding portion thereof, that binds human CTLA4, wherein the antibody or portion is first administered more than one day and less than twenty-eight days following administration of the hormonal therapy agent, and wherein the amounts are effective in combination for the treatment.

In one embodiment, the antibody or portion thereof is administered more than two days following administration of the hormonal therapy agent.

In another embodiment, the antibody or portion thereof is administered less than twenty-one days following administration of the hormonal therapy agent.

In a further embodiment, administration of the hormonal therapy agent terminates prior to the first administration of the antibody or portion thereof.

In yet another embodiment, the hormonal therapy agent is selected from the group consisting of an anti-androgen, a gonadotropin-releasing hormone (GnRH) antagonist, and a luteinizing hormone-releasing hormone (LH-RH) agonist.

In another embodiment, the cancer is selected from a hormone-dependent cancer and a hormone-independent cancer.

In one embodiment, the cancer is hormone-independent and the administration of hormonal therapy terminates prior to the first administration of the antibody or portion thereof.

In another embodiment, the antibody is administered according to a regimen selected from administering about 10 mg/kg every twenty-eight days and administering about 15 mg/kg every three months.

In another embodiment, the anti-CTLA4 antibody, or antigen-binding portion thereof, is at least one antibody selected from the group consisting of:
(a) a human antibody having a binding affinity for CTLA4 of about 10⁻⁸ or greater, and which inhibits binding between CTLA4 and B7-1, and binding between CTLA4 and B7-2;
(b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab;
(c) a human antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab;
(d) a human antibody having the amino acid sequences of a heavy chain variable region and a light chain variable region of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab;
(e) an antibody, or antigen-binding portion thereof, that competes for binding with CTLA4 with at least one antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab; and
(f) an antibody, or antigen-binding portion thereof, that cross-competes for binding with CTLA4 with at least one antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab.

In another embodiment, the antibody is a human antibody having the heavy and light chain amino acid sequences of antibody ticilimumab.

In yet another embodiment, the antibody comprises a heavy chain and a light chain wherein the amino acid sequences of the heavy chain variable region of the heavy chain and the light chain variable region of the light chain are selected from the group consisting of:
(a) the amino acid sequence of SEQ ID NO:3 and the amino acid sequence of SEQ ID NO:9;
(b) the amino acid sequence of SEQ ID NO:15 and the amino acid sequence of SEQ ID NO:21;
(c) the amino acid sequence of SEQ ID NO:27 and the amino acid sequence of SEQ ID NO:33;
(d) the amino acid sequence encoded by the nucleic acid sequence of SEQ ID NO:1 and the amino acid sequence encoded by the nucleic acid sequence of SEQ ID NO:7;
(e) the amino acid sequence encoded by the nucleic acid sequence of SEQ ID NO:13 and the amino acid sequence encoded by the nucleic acid sequence of SEQ ID NO:19;
(f) the amino acid sequence encoded by the nucleic acid sequence of SEQ ID NO:25 and the amino acid sequence encoded by the nucleic acid sequence of SEQ ID NO:31;
(g) the amino acid sequence of a heavy chain variable region and a light chain variable region of antibody ipilimumab.

In one embodiment, the antibody, or antigen-binding portion thereof, is an antibody selected from the group consisting of:
(a) an antibody having a heavy chain variable region comprising the amino acid sequences set forth in SEQ ID NO:4, SEQ ID NO:5, and SEQ ID NO:6, and further having a light chain variable region comprising the amino acid sequences set forth in SEQ ID NO:10, SEQ ID NO:11 and SEQ ID NO:12;
(b) an antibody having a heavy chain variable region comprising the amino acid sequences set forth in SEQ ID NO:16, SEQ ID NO:17, and SEQ ID NO:18, and further having a light chain variable region comprising the amino acid sequences set forth in SEQ ID NO:22, SEQ ID NO:23 and SEQ ID NO:24;
(c) an antibody having a heavy chain variable region comprising the amino acid sequences set forth in SEQ ID NO:28, SEQ ID NO:29, and SEQ ID NO:30, and further having a light chain variable region comprising the amino acid sequences set forth in SEQ ID NO:34, SEQ ID NO:35 and SEQ ID NO:36; and
(d) an antibody having a heavy chain variable region comprising the amino acid sequences of the heavy chain CDR1, CDR2, and CDR3 of antibody ipilimumab, further having a light chain variable region comprising the amino acid sequences of the light chain CDR1, CDR2, and CDR3 of antibody ipilimumab.

The invention includes a method for the treatment of a hormone-independent prostate cancer in a patient in need of such treatment. The method comprises administering to the patient an amount of an antibody that binds human CTLA4, or antigen-binding portion thereof, and an amount of a hormonal therapy agent, wherein the hormonal therapy agent is administered in multiple doses for a time greater than one month and wherein the antibody, or portion thereof, is administered during the period of administration of the hormonal therapy agent, and wherein the amounts are effective in combination for the treatment.

In one embodiment, the hormonal therapy agent is administered over a period greater than two months.

In another embodiment, the method further comprises administering multiple doses of the antibody, or portion thereof, over a period greater than one month that overlaps with the period of administration of the hormonal therapy agent.

In another embodiment, the period of administration of the antibody, or portion thereof, and the period of administration of the hormonal therapy agent overlap by more than two months.

In yet another embodiment, the multiple doses of the antibody, or portion thereof, and the period of administration of the hormonal therapy agent overlap by more than six months.

The invention includes a method for the treatment of hormone-dependent prostate cancer in a patient in need of such treatment. The method comprises co-administering to the patient a therapeutically effective amount of an anti-CTLA4 antibody, or antigen-binding portion thereof, and a therapeutically effective amount of at least two hormonal therapy agents, wherein the agent is selected from the group consisting of an anti-androgen, a gonadotropin-releasing hormone (GnRH) antagonist, and a luteinizing hormone-releasing hormone (LH-RH) agonist.

In one embodiment, the anti-androgen is bicalutamide and the agonist is leuprolide.

The invention includes a pharmaceutical composition for treatment of prostate cancer. The composition comprises a therapeutically effective amount of an anti-CTLA4 antibody, or antigen-binding portion thereof, and a therapeutically effective amount of at least two hormonal therapy agents, wherein the hormonal therapy agent is selected from the group consisting of an anti-androgen, a gonadotropin-releasing hormone (GnRH) antagonist, and a luteinizing hormone-releasing hormone (LH-RH) agonist.

### BRIEF DESCRIPTION OF THE DRAWINGS

The foregoing summary, as well as the following detailed description of the invention, will be better understood when read in conjunction with the appended drawings. For the purpose of illustrating the invention there are shown in the drawings embodiment(s) which are presently preferred. It should be understood, however, that the invention is not limited to the precise arrangements and instrumentalities shown.

In the drawings:

FIG. 1, comprising FIGS. 1A-1D, shows the nucleotide and amino acid sequences for anti-CTLA4 antibody 4.1.1. FIG. 1A shows the full length nucleotide sequence for the 4.1.1 heavy chain (SEQ ID NO:1). FIG. 1B shows the full length amino acid sequence for the 4.1.1 heavy chain (SEQ ID NO:2), and the amino acid sequence for the 4.1.1 heavy chain variable region (SEQ ID NO:3) designated between brackets “[ ]” and the signal peptide sequence is indicated at the amino terminus outside the “[” bracket. The amino acid sequence of each 4.1.1 heavy chain CDR is underlined. The CDR sequences are as follows: CDR1: GFTFSSHGMH (SEQ ID NO:4); CDR2: VIWYDGRNKYYADSV (SEQ ID NO:5); and CDR3: GGHFGPFDY (SEQ ID NO:6). FIG. 1C shows the nucleotide sequence for the 4.1.1 light chain (SEQ ID NO:7). FIG. 1D shows the amino acid sequence of the full length 4.1.1 light chain (SEQ ID NO:8), and the variable region as indicated between brackets “[ ]” (SEQ ID NO:9) and the signal peptide sequence is indicated at the amino terminus outside the “[” bracket. The amino acid sequence of each CDR is indicated as follows: CDR1: RASQSISSSFLA (SEQ ID NO:10); CDR2: GASSRAT (SEQ ID NO:11); and CDR3: QQYGTSPWT (SEQ ID NO:12).

FIG. 2, comprising FIGS. 2A-2D, shows the nucleotide and amino acid sequences for anti-CTLA4 antibody 4.13.1. FIG. 2A shows the full length nucleotide sequence for the 4.13.1 heavy chain (SEQ ID NO:13). FIG. 2B shows the full length amino acid sequence for the 4.13.1 heavy chain (SEQ ID NO:14), and the amino acid sequence for the 4.13.1 heavy chain variable region (SEQ ID NO:15) designated between brackets “[ ]”. The amino acid sequence of each 4.13.1 heavy chain CDR is underlined. The CDR sequences are as follows: CDR1: GFTFSSHGIH (SEQ ID NO:16); CDR2: VIWYDGRNKDYADSV (SEQ ID NO:12); and CDR3: VAPLGPLDY (SEQ ID NO:18). FIG. 2C shows the nucleotide sequence for the 4.13.1 light chain (SEQ ID NO:19). FIG. 2D shows the amino acid sequence of the full length 4.13.1 light chain (SEQ ID NO:20), and the variable region as indicated between brackets “[ ]” (SEQ ID NO:21). The amino acid sequence of each CDR is indicated as follows: CDR1: RASQSVSSYLA (SEQ ID NO:22); CDR2: GASSRAT (SEQ ID NO:23); and CDR3: QQYGRSPFT (SEQ ID NO:24).

FIG. 3, comprising FIGS. 3A-3D, shows the nucleotide and amino acid sequences for anti-CTLA4 antibody 11.2.1, now referred to as ticilimumab. FIG. 3A shows the full length nucleotide sequence for the 11.2.1 heavy chain (SEQ ID NO:25). FIG. 3B shows the full length amino acid sequence for the 11.2.1 heavy chain (SEQ ID NO:26), and the amino acid sequence for the 11.2.1 heavy chain variable region (SEQ ID NO:27) designated between brackets “[ ]”. The amino acid sequence of each 11.2.1 heavy chain CDR is underlined. The CDR sequences are as follows: CDR1: GFTFSSYGMH (SEQ ID NO:28); CDR2: VIWYDGSNKYYADSV (SEQ ID NO:29); and CDR3: DPRGATLYYYYYGMDV (SEQ ID NO:30). FIG. 3C shows the nucleotide sequence for the 11.2.1 light chain (SEQ ID NO:31). FIG. 3D shows the amino acid sequence of the full length 11.2.1 light chain (SEQ ID NO:32), and the variable region as indicated between brackets “[ ]” (SEQ ID NO:33). The amino acid sequence of each CDR is indicated as follows: CDR1: RASQSINSYLD (SEQ ID NO:34); CDR2: AASSLQS (SEQ ID NO:35); and CDR3: QQYYSTPFT (SEQ ID NO:36).

FIG. 4 depicts a photograph demonstrating effect of combination antibody and hormonal therapy neoadjuvant therapy on prostate tissue.

### DETAILED DESCRIPTION OF THE INVENTION

The invention relates in various embodiments to uses of anti-CTLA4 antibodies in combination with at least one hormonal therapy agent to treat prostate cancer in a patient in need of such treatment.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

Preferred amino acid substitutions are those which: (1) reduce susceptibility to proteolysis, (2) reduce susceptibility to oxidation, (3) alter binding affinity for forming protein complexes, and (4) confer or modify other physicochemical or functional properties of such analogs. Analogs comprising substitutions, deletions, and/or insertions can include various muteins of a sequence other than the specified peptide sequence. For example, single or multiple amino acid substitutions (preferably conservative amino acid substitutions) may be made in the specified sequence (preferably in the portion of the polypeptide outside the domain(s) forming intermolecular contacts, e.g., outside of the CDRs). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

Sequence similarity for polypeptides is typically measured using sequence analysis software. Protein analysis software matches similar sequences using measures of similarity assigned to various substitutions, deletions and other modifications, including conservative amino acid substitutions. For instance, Genetics Computer Group (GCG available from Genetics Computer Group, Inc.), also referred to as the Wisconsin Package, is an integrated software package of over 130 programs for accessing, analyzing and manipulating nucleotide and protein sequences. GCG contains programs such as “Gap” and “Bestfit” which can be used with default parameters to determine sequence similarity, homology and/or sequence identity between closely related polypeptides, such as homologous polypeptides from different species of organisms or between a wild type protein and a mutein thereof. See, e.g., GCG version 6.1, version 9.1, and version 10.0.

Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997); herein incorporated by reference.

An intact “antibody” comprises at least two heavy (H) chains and two light (L) chains inter-connected by disulfide bonds. See generally, Fundamental Immunology, Ch. 7 (Paul, W., ed., 2nd ed. Raven Press, N.Y. (1989)) (incorporated by reference in its entirety for all purposes). Each heavy chain is comprised of a heavy chain variable region (HCVR or V_{H}) and a heavy chain constant region (C_{H}). The heavy chain constant region is comprised of three domains, CH1, CH2 and CH3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

The term “antigen-binding portion” of an antibody (or simply “antibody portion”), as used herein, refers to one or more fragments of an antibody that retain the ability to specifically bind to an antigen (e.g., CTLA4). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)); see e.g., Bird et al. Science 242:423-426 (1988) and Huston et al. Proc. Natl. Acad. Sci. USA 85:5879-5883 (1988)). Such single chain antibodies are also intended to be encompassed within the term “antigen-binding portion” of an antibody. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. Proc. Natl. Acad. Sci. USA 90:6444-6448 (1993); Poijak et al. Structure 2:1121-1123 (1994)).

Still further, an antibody or antigen-binding portion thereof may be part of larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov et al. Human Antibodies and Hybridomas 6:93-101 (1995)) and use of a cysteine residue, a marker peptide and a C-terminal polyhistidine tag to make bivalent and biotinylated scFv molecules (Kipriyanov et al. Mol. Immunol. 31:1047-1058 (1994)). Other examples include where one or more CDRs from an antibody are incorporated into a molecule either covalently or noncovalently to make it an immunoadhesin that specifically binds to an antigen of interest, such as CTLA4. In such embodiments, the CDR(s) may be incorporated as part of a larger polypeptide chain, may be covalently linked to another polypeptide chain, or may be incorporated noncovalently. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Where an “antibody” is referred to herein with respect to the present invention, it should be understood that an antigen-binding portion thereof may also be used. An antigen-binding portion competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide. In embodiments having one or more binding sites, the binding sites may be identical to one another or may be different.

The terms “human antibody” or “human sequence antibody”, as used interchangeably herein, include antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include “chimeric” antibodies in which CDR sequences derived from the germline of another mammalian species, such as a mouse, have been grafted onto human framework sequences (i.e., “humanized” or PRIMATIZED™ antibodies).

The term “chimeric antibody” as used herein means an antibody that comprises regions from two or more different antibodies. In one embodiment, one or more of the CDRs are derived from a human anti-CTLA4 antibody. In another embodiment, all of the CDRs are derived from a human anti-CTLA4 antibody. In another embodiment, the CDRs from more than one human anti-CTLA4 antibodies are combined in a chimeric human antibody. For instance, a chimeric antibody may comprise a CDR1 from the light chain of a first human anti-CD40 antibody, a CDR2 from the light chain of a second human anti-CTLA4 antibody and a CDR3 and CDR3 from the light chain of a third human anti-CTLA4 antibody, and the CDRs from the heavy chain may be derived from one or more other anti-CD40 antibodies. Further, the framework regions may be derived from one of the same anti-CTLA4 antibodies or from one or more different human(s).

Moreover, as discussed previously herein, chimeric antibody includes an antibody comprising a portion derived from the germline sequences of more than one species.

By the term “effective amount”, or “therapeutically effective amount,” as used herein, is meant an amount that when administered to a mammal, preferably a human, mediates a detectable therapeutic response compared to the response detected in the absence of the compound. A therapeutic response, such as, but not limited to, inhibition of and/or decreased tumor growth, tumor size, metastasis, and the like, can be readily assessed by a plethora of art-recognized methods, including, e.g., such methods as disclosed herein.

The skilled artisan would understand that the effective amount of the compound or composition administered herein varies and can be readily determined based on a number of factors such as the disease or condition being treated, the stage of the disease, the age and health and physical condition of the mammal being treated, the severity of the disease, the particular compound being administered, and the like.

By the term “co-administration,” is meant that the hormone therapy and antibody therapy are administered to the patient substantially contemporaneously of each other. That is, the hormone therapy and antibody therapy are administered on the same day, or there is no more than 4 months between administration of the final dose of hormone therapy in the treatment cycle (e.g., a high dose of leuprolide acetate for depot suspension which is typically administered every 4 months such that exposure to the hormone therapy may persist) and the administration of the antibody, or there is no more than 3 months between administration of a dose of antibody and the administration of hormone therapy.

By the term “compete”, as used herein with regard to an antibody, is meant that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). For instance, cross-competing antibodies can bind to the epitope, or portion of the epitope, to which the antibodies used in the invention bind. Use of both competing and cross-competing antibodies is encompassed by the present invention. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

“Hormonal therapy,” or “androgen suppression,” as the terms are used interchangeably herein, encompass any method comprising administration of an agent or compound whereby the level of an androgen (male steroid hormone, such as, but not limited to, testosterone and dihydrotestosterone) is detectably decreased compared with the level of the androgen in the absence of, or prior to, the administration of the agent or compound. Hormonal therapy includes, but is not limited to, use of anti-androgens, gonadotropin-releasing hormone antagonists, and luteinizing hormone-releasing hormone agonists, and combinations thereof.

“Anti-androgen”, as the term is used herein, refers to any agent or compound that detectably blocks an androgen receptor on a prostate cell thereby decreasing the level of androgens in a mammal compared to the level of androgens in the mammal prior to administration of the agent or compound and/or compared with the level of androgens in an otherwise identical mammal to which the agent or compound is not administered. Preferably, the mammal is a human.

An anti-androgen can be “non-steroidal” or “steroidal” as these terms are understood in the art and used herein. That is, a non-steroidal anti-androgen (e.g., bicalutamide (CASODEX), nilutamide (NILANDRON), flutamide (EULEXIN, DROGENIL), and the like) competitively binds to the androgen receptor on a prostate cell, inhibiting the stimulatory effect of androgens on the prostate. Steroidal anti-androgens (e.g., megestrol (MEGACE), and cyproterone (ANDROCUR) not only inhibit androgen binding at the cellular receptor, they also slow release of LH from the pituitary gland.

“Gonadotropin-releasing hormone (GnRH) antagonist”, as used herein, refers to an agent that binds to GnRH (also known as LH-RH) receptors in the pituitary gland thereby blocking the receptors without causing gonadotropin release. GnRN antagonists include, e.g., abarelix (PLENAXIS) and histrelin (SUPPRELIN). GnRH antagonists, unlike LH-RH agonists, do not cause a detectable testosterone surge.

The term “luteinizing hormone-releasing hormones (LH-RH) agonist”, means an agent or compound that detectably decreases pituitary gland production of at least one hormone that otherwise stimulates testosterone production compared with the level of pituitary production of such hormone in the absence of, or prior to, administration of the LH-RH agonist. A LH-RH agonist, as the term used herein, is an agent that causes continuous super-stimulation of the LH-RH receptor and internalization of the LH-RH receptor complex thereby rendering the pituitary cell refractory to further stimulation due to decreased presence of the receptor on the cell. This, in turn, results in a decrease in LH and subsequent secretion of testosterone. LH-RH agonists include, but are not limited to, leuprorelin (leuprolide; LUPRON, ELIGARD), goserelin (ZOLADEX), buserelin (SUPREFACT), tryptorelin (DECAPEPTYL), and the like.

By the term “maximal androgen blockade,” is meant combination of at least one anti-androgen with at least one LH-RH agonist or orchiectomy to block androgen hormones. Any combination of these methods and compounds, including multiple anti-androgens, multiple LH-HR agonists, and any permutation thereof, is encompassed in the term.

“Instructional material,” as that term is used herein, includes a publication, a recording, a diagram, or any other medium of expression which can be used to communicate the usefulness of the compound, combination, and/or composition of the invention in the kit for affecting, alleviating or treating the various diseases or disorders recited herein. Optionally, or alternately, the instructional material can describe one or more methods of alleviating the diseases or disorders in a cell, a tissue, or a mammal, including as disclosed elsewhere herein.

The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

Except when noted, the terms “patient” or “subject” are used interchangeably and refer to mammals such as human patients and non-human primates, as well as veterinary subjects such as rabbits, rats, and mice, and other animals. Preferably, patient refers to a human.

Conventional notation is used herein to portray polypeptide sequences: the left-hand end of a polypeptide sequence is the amino-terminus; the right-hand end of a polypeptide sequence is the carboxyl-terminus.

By the phrase “specifically binds,” as used herein, is meant a compound, e.g., a protein, a nucleic acid, an antibody, and the like, which recognizes and binds a specific molecule, but does not substantially recognize or bind other molecules in a sample. For instance, an antibody or a peptide inhibitor which recognizes and binds a cognate ligand (e.g., an anti-CTLA4 antibody that binds with its cognate antigen, CTLA4) in a sample, but does not substantially recognize or bind other molecules in the sample. Thus, under designated assay conditions, the specified binding moiety (e.g., an antibody or an antigen-binding portion thereof) binds preferentially to a particular target molecule and does not bind in a significant amount to other components present in a test sample. A variety of assay formats may be used to select an antibody that specifically binds a molecule of interest. For example, solid-phase ELISA immunoassay, immunoprecipitation, BIAcore and Western blot analysis are used to identify an antibody that specifically reacts with CTLA4. Typically a specific or selective reaction will be at least twice background signal or noise and more typically more than 10 times background, even more specifically, an antibody is said to “specifically bind” an antigen when the equilibrium dissociation constant (K_{D}) is ≦1 μM, preferably ≦100 nM and most preferably ≦10 nM.

The term “K_{D}” refers to the equilibrium dissociation constant of a particular antibody-antigen interaction.

As used herein, “substantially pure” means an object species is the predominant species present (i.e., on a molar basis it is more abundant than any other individual species in the composition), and preferably a substantially purified fraction is a composition wherein the object species (e.g., an anti-CTLA4 antibody) comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

As used herein, to “treat” means reducing the frequency with which symptoms of a disease (i.e., tumor growth and/or metastasis, or other effect mediated by the numbers and/or activity of immune cells, and the like) are experienced by a patient. The term includes the administration of the compounds or agents of the present invention to prevent or delay the onset of the symptoms, complications, or biochemical indicia of a disease (e.g., elevation of PSA level), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

I. Combination Therapy

The invention relates to methods described below for the administration of an antibody that binds human CTLA4, or an antigen-binding portion of the antibody, in combination with hormonal therapy to treat prostate cancer. Whether the prostate cancer is hormone-dependent or -independent, combination of CTLA4 blockade with reduction in the level of androgen in a patient according to a method of the invention may provide a synergistic therapeutic benefit as more fully discussed below.

Although the invention encompasses numerous combination therapies wherein the antibody is administered to the patient in combination with at least one prostate tumor hormonal therapeutic agent, the present invention is in no way limited to the exemplified agents, which are set forth herein for illustrative purposes only.

In one embodiment of the invention, an anti-CTLA4 antibody, or antigen-binding portion thereof, is administered more than one day and less than twenty-eight days after an administration of a hormonal therapy agent. The amount of the hormonal therapy agent and the antibody are effective in combination for prostate cancer treatment. Hormone therapy may be administered prior to administration of antibody therapy irrespective of the hormone-dependent status of the tumor. For example, where the tumor is hormone-dependent, the hormone-dependent status has not been assessed but no prior hormone therapy has been administered, or the tumor is known to be hormone-independent (also referred to as “hormone refractory”), an antibody that binds human CTLA4, or an antigen-binding portion of the antibody, is administered more than one day and less than twenty-eight days following administration of the hormonal therapy agent. The agent includes, but is not limited to, an anti-androgen, a gonadotropin-releasing hormone (GnRH) antagonist, and a luteinizing hormone-releasing hormone (LH-RH) agonist, or combination thereof.

In one embodiment, a synergistic or additive effect is mediated by administration of the anti-CTLA4 antibody more than one day after administration of at least one hormone therapy agent. Without wishing to be bound by any particular theory of the invention, hormonal therapy may increase, perhaps by apoptotic or other architectural changes to the prostate, exposure of prostate tumor specific antigen(s) to the immune system such that the immune response to the tumor cell is increased. That is, hormonal therapy may create or increase a source of tumor-specific antigen in the host mediated by tumor cell death which in turn may feed tumor antigen into host antigen presentation pathways. Anti-CTLA4 antibody mediates an increased immune response to the increased levels of tumor-specific antigen in the antigen presentation pathway thereby providing a synergistic therapeutic effect. Other combination therapies that may result in synergy with anti-CTLA4 enhancement of the immune response through cell death release of tumor-specific antigens are radiation, surgery, and hormone therapy, among others.

Further, a decrease in androgen level may increase or prolong an immune response in the patient that may be mediated by a non-tumor-specific effect, e.g., lowering the level of an androgen may mediate an enhanced immune response by a mechanism unrelated to an attack on the tumor. Data disclosed herein suggests that there may be an apparent biological interaction between androgen blockade and the anti-CTLA4 antibody of the invention that leads to a higher severity of diarrhea observed with the antibody alone suggesting increased effectiveness of the combination therapy. Previous studies suggest that androgens may play a role in the immune system. See generally Grossman, Science 227:257-261 (1985) (discussing regulation of the immune system by gonadal steroids); Olsen and Kovacs, Immunologic Res. 23:281-288 (2001) (discussing the effects of androgens on T and B lymphocyte development); Sutherland et al., J. Immunol. 175: 2741-2753 (2005) (noting thymic regeneration following androgen blockade); Tanriverdi et al., J. Clin. Endocrinol. 176:293-304 (2003) (discussing the potential interplay between the immune system and GnRH and sex steroids); and Mercader et al., Proc Natl Acad Sci USA 98:14565-14570 (2001) (noting T cell infiltration of prostate is increased upon androgen withdrawal); Arlen et al., J Urol 174:539-546 (2005) (discussing vaccination using poxvirus vectors expressing prostate antigen, PSA, in combination with hormone therapy).

Without wishing to be bound by any theory of the invention, the combination of androgen blockade and anti-CTLA4 antibody may induce a more robust immunological response within the prostate than expected. Therefore, the combination of androgen blockade using, among others, a combination of leuprolide and bicalutamide, in combination with an anti-CTLA-4 antibody, can provide a potential synergistic effect thereby providing an important novel therapeutic treatment for prostate cancer.

In one embodiment, the antibody therapy is administered less than twenty-eight days after hormonal therapy to a patient who has not received prior hormonal therapy. This may be done where, e.g., the hormone dependent status of the tumor has not yet been assessed. Antibody therapy is administered at least one day and less than twenty-eight days following an administration of hormone therapy. Preferably, the hormonal therapy is continued throughout the course of the antibody therapy.

In another embodiment, the antibody, or portion thereof, is administered less than twenty-one days following administration of the hormonal therapy agent. Further, in one embodiment of the present invention, the antibody, or antigen-binding portion thereof, is administered more than two days after administration of the hormonal therapy agent.

In yet another embodiment, the antibody is administered more than one day and less than twenty-eight days following administration of the hormonal therapy agent, where administration of hormonal therapy terminates prior to administration of the antibody. That is, the patient does not receive additional hormonal therapy once the antibody is administered; however, multiple doses of the antibody may be administered as desired without further administration of hormonal therapy. Similarly, in another embodiment, the antibody is administered more than two days and less than twenty-one days following administration of the hormonal therapy agent, and administration of the hormonal therapy terminates prior to administration of the antibody.

In one embodiment, the prostate cancer is hormone-dependent, while in another embodiment, the cancer is hormone-independent. Thus, where the antibody is administered more than one day and less than twenty-eight days following administration of the hormonal therapy agent, the prostate cancer may be hormone-dependent or hormone-independent. Such administration may provide a synergistic therapeutic benefit regardless of hormone status of the tumor.

Where the tumor is hormone-dependent, the hormonal therapy agent can be administered. Antibody therapy is administered to the patient after at least one day and less than twenty-eight days from the administration of a dose of hormone therapy, hormone therapy may continue following administration of the antibody. According to one theory of the invention, this therapy may provide a benefit to the patient in that the therapy may provide increased exposure of tumor-specific antigen(s) and/or decrease any immune down-regulatory effect of androgens such that an immune response to the tumor is elicited and/or prolonged, thereby providing a therapeutic benefit to the patient. Termination of hormonal therapy has been associated with tumor response, e.g., tumor regression, stabilization or decline of PSA level, decrease bone pain, and the like, suggesting that ceasing the hormonal therapy is associated with a paradoxical change in the control of tumor growth that may have a synergistic effect when combined with subsequent CTLA4 antibody therapy, thereby providing a therapeutic benefit.

In another embodiment, where the tumor is hormone-independent, hormone therapy comprising at least one hormonal therapy agent is co-administered with an anti-CTLA4 antibody. Even where a tumor is hormone-independent, administration of androgen-reducing therapy may provide a synergistic or additive therapeutic effect where co-administered with antibody therapy. As more fully discussed previously, without wishing to be bound by any particular theory of the invention, it may be that hormonal therapy that reduces androgen levels mediates an increased exposure of tumor antigens to the immune system and/or reduces any immunosuppressive effect that the androgens might otherwise mediate. Alternatively, it may be that hormonal therapy that reduces androgen levels mediates an effect on the thymus that enhances the ability of the immune system to mount an adaptive immune response [see Sutherland et al., J Immunol. 175:2741-2753 (2005)). However, the invention encompasses providing a therapeutic benefit to a patient by any mechanism by administering such combination therapy.

In another embodiment, the antibody therapy is administered prior to administration of hormonal therapy. That is, antibody therapy may be administered once a month, or alternatively, once every two or three months, to its maximal benefit (e.g., tumor regression, decreased bone pain, decrease or stabilization of PSA level, etc.) and then followed by hormonal therapy either in addition to antibody therapy or antibody therapy is stopped and hormone therapy is then administered. This is because, due to the long half life of the antibody and the persistence of T cells effected by the prior administration of the antibody therapy, subsequent additional HT, or substitution of antibody therapy with HT, may alter the nature and/or enhance the antibody-initiated immune response. Thus, the invention encompasses administration of antibody therapy first, preferably for a protracted period (e.g., one month, more preferably, two months, even more preferably, three months, and yet more preferably, four months of antibody therapy), either followed by co-administration of hormonal therapy or substitution of the antibody therapy with hormonal therapy.

In one embodiment, the antibody therapy is neoadjuvant therapy administered prior to surgery. Following surgery, hormonal therapy is administered, either instead of continued antibody therapy or along with continued antibody therapy. This is because, as discussed previously, administration of antibody therapy prior to hormonal therapy, either followed by hormonal therapy in the absence of continued antibody therapy or where the hormonal therapy is administered in addition to the antibody therapy following prostatectomy may provide a synergistic therapeutic effect to a patient.

As noted previously herein, many hormonal therapy agents are known in the art, such that the invention encompasses, but is not limited to, use of exemplary anti-androgens, GnRH antagonists, and LH-RH agonists. Anti-androgens include, e.g., non-steroidal anti-androgens such as, but not limited to, bicalutamide (CASODEX), nilutamide (NILANDRON), flutamide (EULEXIN, DROGENIL), and steroidal anti-androgens, including megestrol (MEGACE), and cyproterone (ANDROCUR). GnRH antagonists include, among others, abarelix (PLENAXIS) and histrelin (SUPPRELIN). LH-RH agonists include, e.g., leuprorelin (leuprolide; LUPRON, ELIGARD), goserelin (ZOLADEX), buserelin (SUPREFACT), tryptorelin (DECAPEPTYL), and the like.

In another embodiment, treatment of hormone-independent prostate cancer comprises administering an antibody, or antigen-binding portion thereof, and a hormonal therapy agent to a patient, where the hormonal therapy is administered in multiple doses for a time greater than one month and where the antibody, or antigen-binding portion, is administered during the period of administration of the hormonal therapy. Further, the amounts are effective in combination for treatment of the prostate cancer. As discussed above, according to one theory of the invention, even where prostate cancer is hormone-independent, combination of hormonal therapy with antibody therapy may mediate exposure of tumor antigens and/or decrease any immunosuppressive effect(s) of androgens such that hormonal therapy combined with CTLA4 blockade can provide an enhanced and/or prolonged immune response when compared to either therapy alone.

In one embodiment, the hormonal therapy agent is administered in multiple doses over a period greater than two months. Moreover, in another embodiment, during the period of greater than two months of administration of the hormonal therapy, multiple doses of the antibody are administered over a period of greater than one month. The period of administration of multiple doses of antibody over a period of more than one month may overlap with the period of administration of the hormonal therapy. In another embodiment, the period of administration of antibody and the period of administration of hormonal therapy may overlap by more than two months. Further, in yet another embodiment, the period of administration of antibody and the period of administration of hormonal therapy overlap by more than six months. As would be appreciated by one skilled in the art provided with the disclosure herein, the period of administration of hormonal therapy, as well as the period of administration of antibody, may overlap and be adjusted as indicated to provide a therapeutic benefit.

In another embodiment, where prior hormonal therapy has been discontinued because the tumor is considered hormone-independent, hormonal therapy is re-administered (i.e., using either the same or a different hormonal therapy agent as was administered previously) in combination with the anti-CTLA4 antibody. In this instance, hormonal therapy comprises administering at least one hormonal therapy agent, e.g., an anti-androgen and a LH-RH agonist, an anti-androgen and a GnRH antagonist and an anti-CTLA4 antibody (e.g., 4.1.1, 4.13.1, 6.1.1, ticilimumab, ipilimumab), or an antigen-binding portion thereof. More preferably, the hormonal therapy agent includes, but is not limited to, bicalutamide, flutamide, nilutamide, and leuprolide, and is administered with the antibody over a period of greater than one month. In another embodiment, multiple doses of the antibody and the hormonal therapy agent are administered and the period of administration of the antibody and the hormonal therapy overlap over a period of at least one month.

In yet another embodiment, hormonal therapy comprising at least two hormone therapy agents is co-administered with antibody therapy to a patient where the tumor is hormone-dependent. That is, hormonal therapy comprising at least two hormonal therapy agents, thereby providing maximal androgen ablation therapy, is administered to a hormone-dependent prostate cancer patient, where the tumor-dependent status of the tumor has been determined and/or where the status has not been determined, but where the patient has not received prior hormone therapy. Without wishing to be bound by any theory of the invention, maximal androgen ablation hormonal therapy co-administered with anti-CTLA4 antibody therapy may provide a benefit to the patient in that the combination may provide increased exposure of tumor-specific antigen(s) and/or decrease any immune-suppressive effect(s) of androgens such that an immune response to the tumor is elicited and/or prolonged thereby providing a therapeutic benefit to the patient.

Thus, in this embodiment of the invention, where the prostate cancer is hormone-dependent, an effective amount of at least two hormonal therapy agents independently selected from an anti-androgen, a GnRH antagonist, and a LH-RH agonist, and an effective amount of an anti-CTLA4 antibody, or an antigen-binding portion thereof, are administered to the patient. Administration of the hormonal therapy agents and the antibody may overlap and each may be administered in multiple doses over a period that may, but need not, overlap. The length of the course of treatment may be adjusted as indicated and as would be understood by one skilled in the art of cancer treatment.

Administration of the antibody and the hormonal therapy agents, either contemporaneously or sequentially, encompasses administering a pharmaceutical composition comprising both the anti-CTLA4 antibody and one or more additional hormonal therapy agents, and administering two or more separate pharmaceutical compositions, one comprising the anti-CTLA4 antibody and the other(s) comprising the additional hormonal therapy agents. Further, although co-administration or combination (conjoint) therapy encompasses administering at the same time as one another, it also encompasses simultaneous, sequential or separate dosing of the individual components of the treatment. Additionally, where an antibody is administered intravenously and the hormonal therapy agent(s) is/are administered orally (e.g., bicalutamide, and the like), or by subcutaneous or intramuscular injection (e.g., leuprolide), it is understood that the combination may be administered as separate pharmaceutical compositions.

Where the treatment comprises co-administering a combination of an anti-CTLA4 antibody and at least two hormone therapy agents, agents may be, among many others, an anti-androgen, a gonadotropin-releasing hormone (GnRH) antagonist, and a luteinizing hormone-releasing hormone (LH-RH) agonist. Many such hormonal therapy agents are known in the art, such that the invention encompasses, but is not limited to, exemplary compounds in each of the afore-mentioned hormonal therapeutic agent classes. More particularly, anti-androgens including, e.g., non-steroidal anti-androgens include bicalutamide (CASODEX), nilutamide (NILANDRON), flutamide (EULEXIN, DROGENIL), and the like. Steroidal anti-androgens include megestrol (MEGACE), and cyproterone (ANDROCUR). Gonadotropin-releasing hormone (GnRH) antagonists include, among others, abarelix (PLENAXIS) and histrelin (SUPPRELIN). Further, luteinizing hormone-releasing hormones (LH-RH) agonists include, e.g., leuprorelin (leuprolide; LUPRON, ELIGARD), goserelin (ZOLADEX), buserelin (SUPREFACT), tryptorelin (DECAPEPTYL), and the like. In one embodiment, the anti-CTLA4 antibody is co-administered with an anti-androgen (bicalutamide) and a LH-RH agonist (leuprolide), but any combination of at least two hormonal therapeutic agents, as well as a single hormonal therapy agent, and an anti-CTLA4 antibody is encompassed by the invention.

As noted elsewhere herein, leuprolide is a member of the class of luteinizing hormone-releasing hormone (LH-RH) antagonists. Leuprolide is useful for treatment of, among other things, prostate cancer in men, endometriosis and fibroids in women, and central precocious puberty in children. Combination of anti-CTLA4 antibody and leuprolide, in further combination with bicalutamide, is useful for treatment of prostate cancer. The invention encompasses use of an anti-CTLA4 antibody in combination with anti-androgen therapy, such as, but not limited to, leuprolide and bicalutamide, as a neoadjuvant, adjuvant, and/or first line and second line for non-metastatic rising PSA (biochemical recurrence), and/or metastatic prostate cancer. Preferably, the combination is used as a neoadjuvant therapy for prostate cancer. More particularly, neoadjuvant therapy is administered prior to any subsequent treatment, e.g., the hormonal therapy and antibody therapy combination is administered prior to prostatectomy. Additionally, the combination therapy, or the antibody therapy as a single agent, can be continued following prostatectomy as exemplified elsewhere herein.

The methods of the invention can be carried out as a neoadjuvant therapy prior to surgery, radiation therapy, or any other treatment, in order to sensitize the tumor cells or to otherwise confer a therapeutic benefit to the patient. However, the invention is not limited to the neoadjuvant setting. Rather, the methods of the invention can be used along the entire disease and treatment continuum, e.g., but not limited to, adjuvant, rising PSA (prior to the appearance of clinically evident metastases), and first-line, second-line and/or third-line therapy for prostate cancer.

The present invention may be further combined with additional agents and therapies, e.g., chemotherapy, surgery, radiotherapy, transplantation, and the like, to treat a patient. That is, the patient may be subjected to additional chemotherapy with agents well-known, such as, but not limited to, growth factor inhibitors, biological response modifiers, alkylating agents, intercalating antibiotics, vinca alkaloids, immunomodulators, taxanes, selective estrogen receptor modulators (SERMs), such as, but not limited to, lasofoxifene, and angiogenesis inhibitors.

Therapeutic agents are numerous and have been described in, for instance, U.S. Patent Application Publication No. 2004/0005318, No. 2003/0086930, No. 2002/0086014, and International Publication No. WO 03/086459, all of which are incorporated by reference herein, among many others. Such therapeutic agents include, but are not limited to, topoisomerase I inhibitors; other antibodies (bevacizumab, cetuximab, panitumumab, rituximab, trastuzumab, pertuzumab, anti-IGF-1R, anti-MAdCAM, anti-CD40, anti-4-1BB, and the like); chemotherapeutic agents such as, but not limited to, imatinib (GLEEVEC), SU11248 (SUTENT), SU12662, SU14813; BAY 43-9006, indoleamine-2,3,-dioxygenase (IDO) inhibitors, selective estrogen receptor modulators (SERMs; e.g., lasofoxifene), taxanes, vinca alkaloids, temozolomide, angiogenesis inhibitors, EGFR inhibitors, VEGF inhibitors, erbB2 receptor inhibitors, anti-proliferative agents (e.g., farnesyl protein transferase inhibitors, and αvβ3 inhibitors, αvβ5 inhibitors, p53 inhibitors, and the like), immunomodulators, cytokines, tumor vaccines; tumor-specific antigens; heat shock protein-based tumor vaccines; dendritic and stem cell therapies; alkylating agents, folate antagonists; pyrimidine antagonists; anthracycline antibiotics; platinum compounds; costimulatory molecules (e.g., CD4, CD25, PD-1, B7-H3, 4-1BB, OX40, ICOS, CD30, HLA-DR, MHCII, and LFA, and agonist antibodies thereto).

As mentioned above, the methods of the invention may be further combined with transplantation, e.g., stem cell transplantation, to provide a therapeutic benefit to a patient afflicted with prostate cancer. Stem cell transplantation may be performed according to the methods known in the art and may be allogeneic or autologous stem cell transplantation. Additionally, one skilled in the art would appreciate, based upon the disclosure provided herein, that transplantation encompasses adoptive transfer of lymphocytes, either autologous or obtained from an HLA-matched donor. Where the method comprises stem cell transplant, the first dose of the antibody-hormone therapy agent combination can be administered after the immune system of the mammal has recovered from transplantation, for example, in the period of from one to 12 months post transplantation. In certain embodiments, the first dose is administered in the period of from one to three, or one to four months post transplantation. Transplantation methods are described many treatises, including Appelbaum in Harrison's Principles of Internal Medicine, Chapter 14, Braunwald et al., Eds., 15^{th} ed., McGraw-Hill Professional (2001), which is hereby incorporated herein by reference.

The present invention also encompasses the administration of other therapeutic agents in addition to anti-CTLA4 antibody and hormonal therapy agents. Such therapeutic agents include analgesics, cancer vaccines, anti-vascular agents, anti-proliferative agents, anti-emetic agents, and anti-diarrheal agents. Preferred anti-emetic agents include ondansetron hydrochloride, granisetron hydrochloride, and metoclopramide. Preferred anti-diarrheal agents include diphenoxylate and atropine (LOMOTIL), loperamide (IMMODIUM), and octreotide (SANDOSTATIN).

In another embodiment, the invention includes administering an agent with anti-diarrheal effect wherein the agent is indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

II. Anti-CTLA4 Antibodies

In one embodiment, the CTLA4 antibody comprises a heavy chain wherein the amino acid sequence of the V_{H} comprises the amino acid sequences set forth in SEQ ID NOs:3, 15 and 27. In yet another embodiment, the V_{L} of the CTLA4 antibody comprises the amino acid sequences set forth in SEQ ID NOs:9, 21 and 33. More preferably, the V_{H} and V_{L} regions of the antibody comprise the amino acid sequences set forth in SEQ ID NO:3 (V_{H} 4.1.1) and SEQ ID NO:9 (V_{L} 4.1.1), respectively; the amino acid sequences set forth in SEQ ID NO:15 (V_{H} 4.13.1) and SEQ ID NO:21 (V_{L} 4.13.1), respectively; and the amino acid sequences set forth in SEQ ID NO:27 (V_{H} ticilimumab) and SEQ ID NO:33 (V_{L} ticilimumab), respectively. Most preferably, the antibody is ticilimumab (also known as CP-675,206, which has the heavy and light chain amino acid sequences of antibody ticilimumab).

In yet another embodiment, the amino acid sequence of the heavy chain comprises the amino acid sequence encoded by a nucleic acid comprising the nucleic acid sequences set forth in SEQ ID NOs:1, 13, and 25. In yet another embodiment, the light chain comprises the amino acid sequence encoded by a nucleic acid comprising the nucleic acid sequences set forth in SEQ ID NOs:7, 19 and 31. More preferably, the heavy and light chains comprise the amino acid sequences encoded by nucleic acids comprising the nucleic acid sequences set forth in SEQ ID NO:1 (heavy chain 4.1.1) and SEQ ID NO:7 (light chain 4.1.1), respectively; the nucleic acid sequences set forth in SEQ ID NO:13 (heavy chain 4.13.1) and SEQ ID NO:19 (light chain 4.13.1), respectively; and the nucleic acid sequences set forth in SEQ ID NO:25 (heavy chain ticilimumab) and SEQ ID NO:31 (light chain ticilimumab), respectively.

Furthermore, the antibody can comprise a heavy chain amino acid sequence comprising human CDR amino acid sequences derived from the V_{H} 3-30 or 3-33 gene, or conservative substitutions or somatic mutations therein. It is understood that the V_{H} 3-33 gene encodes from FR1 through FR3 of the heavy chain variable region of an antibody molecule. Thus, the invention encompasses an antibody that shares at least 85%, more preferably, at least 90%, yet more preferably, at least 91%, even more preferably, at least 94%, yet more preferably, at least 95%, more preferably, at least 97%, even more preferably, at least 98%, yet more preferably, at least 99%, and most preferably, 100% identity, with the sequence from FR1 through FR3 of an antibody selected from the group consisting of 3.1.1, 4.1.1, 4.8.1, 4.10.2, 4.13.1, 4.14.3, 6.1.1, ticilimumab, 11.6.1, 11.7.1, 12.3.1.1, 2.9.1.1, ipilimumab, and DP-50.

The antibody can further comprise CDR regions in its light chain derived from the A27 or the O12 gene or it may comprise the CDR regions of an antibody selected from the group consisting of 3.1.1, 4.1.1, 4.8.1, 4.10.2, 4.13.1, 4.14.3, 6.1.1, ticilimumab, 11.6.1, 11.7.1, 12.3.1.1, 2.9.1.1, ipilimumab.

In other embodiments of the invention, the antibody inhibits binding between CTLA4 and B7-1, B7-2, or both. Preferably, the antibody can inhibit binding with B7-1 with an IC₅₀ of about 100 nM or lower, more preferably, about 10 nM or lower, for example about 5 nM or lower, yet more preferably, about 2 nM or lower, or even more preferably, for example, about 1 nM or lower. Likewise, the antibody can inhibit binding with B7-2 with an IC₅₀ of about 100 nM or lower, more preferably, 10 nM or lower, for example, even more preferably, about 5 nM or lower, yet more preferably, about 2 nM or lower, or even more preferably, about 1 nM or lower.

Further, in another embodiment, the anti-CTLA4 antibody has a binding affinity for CTLA4 of about 10⁻⁸, or greater affinity, more preferably, about 10⁻⁹ or greater affinity, more preferably, about 10⁻¹⁰ or greater affinity, and even more preferably, about 10⁻¹¹ or greater affinity.

The anti-CTLA4 antibody includes an antibody that competes for binding with an antibody having heavy and light chain amino acid sequences of an antibody selected from the group consisting of 4.1.1, 6.1.1, ticilimumab, 4.13.1 and 4.14.3. Further, the anti-CTLA4 antibody can compete for binding with antibody ipilimumab.

In another embodiment, the antibody preferably cross-competes with an antibody having a heavy and light chain sequence, a variable heavy and a variable light chain sequence, and/or the heavy and light CDR sequences of antibody 4.1.1, 4.13.1, 4.14.3, 6.1.1. or ticilimumab. For example, the antibody can bind to the epitope to which an antibody that has heavy and light chain amino acid sequences, variable sequences and/or CDR sequences, of an antibody selected from the group consisting of 4.1.1, 4.13.1, 4.14.3, 6.1.1, or ticilimumab binds. In another embodiment, the antibody cross-competes with an antibody having heavy and light chain sequences, or antigen-binding sequences, of ipilimumab.

In another embodiment, the invention is practiced using an anti-CTLA4 antibody that comprises a heavy chain comprising the amino acid sequences of CDR-1, CDR-2, and CDR-3, and a light chain comprising the amino acid sequences of CDR-1, CDR-2, and CDR-3, of an antibody selected from the group consisting of 3.1.1, 4.1.1, 4.8.1, 4.10.2, 4.13.1, 4.14.3, 6.1.1, ticilimumab, 11.6.1, 11.7.1, 12.3.1.1, and 12.9.1.1, or sequences having changes from the CDR sequences selected from the group consisting of conservative changes, wherein the conservative changes are selected from the group consisting of replacement of nonpolar residues by other nonpolar residues, replacement of polar charged residues other polar uncharged residues, replacement of polar charged residues by other polar charged residues, and substitution of structurally similar residues; non-conservative substitutions, wherein the non-conservative substitutions are selected from the group consisting of substitution of polar charged residue for polar uncharged residues and substitution of nonpolar residues for polar residues, additions and deletions.

In a further embodiment of the invention, the antibody contains fewer than 10, 7, 5, or 3 amino acid changes from the germline sequence in the framework or CDR regions. In another embodiment, the antibody contains fewer than 5 amino acid changes in the framework regions and fewer than 10 changes in the CDR regions. In one preferred embodiment, the antibody contains fewer than 3 amino acid changes in the framework regions and fewer than 7 changes in the CDR regions. In a preferred embodiment, the changes in the framework regions are conservative and those in the CDR regions are somatic mutations.

In another embodiment, the antibody shares at least 80%, more preferably, at least 85%, even more preferably, at least 90%, yet more preferably, at least 94%, preferably, at least 95%, more preferably, at least 99%, sequence (e.g., amino acid, nucleic acid, or both) identity or sequence similarity over the heavy and light chain full-length sequences, or over the heavy or the light chain, separately, with the sequences of antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab. Even more preferably, the antibody shares 100% sequence identity or sequence similarity over the heavy chain and the light chain, or with the heavy chain or the light chain, separately, of an antibody selected from antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab.

In another embodiment, the antibody shares at least 80%, more preferably, at least 85%, even more preferably, at least 90%, yet more preferably, at least 94%, more preferably, at least 95%, even more preferably, at least 99%, sequence identity or sequence similarity over the heavy and light chain full-length sequences, or over the heavy or the light chain, separately, with the sequences of germline V_{κ} A27, germline V_{κ} O12, and germline DP50 (which is an allele of the V_{H} 3-33 gene locus). Even more preferably, the antibody shares 100% sequence identity or sequence similarity over the heavy chain sequence of germline DP50 and/or with the light chain sequence of germline A27, or germline O12.

In one embodiment, the antibody shares at least 80%, more preferably, at least 85%, even more preferably, at least 90%, yet more preferably, at least 94%, preferably, at least 95%, more preferably, at least 99%, sequence (e.g., amino acid, nucleic acid, or both) identity or sequence similarity over the heavy and light chain variable region sequences, or over the heavy or the light chain variable region sequence, separately, with the sequences of antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab. Even more preferably, the antibody shares 100% sequence identity or sequence similarity over the heavy chain and the light chain variable region sequences, or with the heavy chain or the light chain sequence, separately, of an antibody selected from antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab.

In another embodiment, the antibody shares at least 80%, more preferably, at least 85%, even more preferably, at least 90%, yet more preferably, at least 94%, more preferably, at least 95%, even more preferably, at least 99%, sequence identity or sequence similarity over heavy chain variable region sequence with the heavy chain variable sequence of heavy germline DP50 (which is an allele of the V_{H} 3-33 gene locus) or with the light chain variable sequence of germline V_{κ} A27, or germline V_{κ} O12. Even more preferably, the antibody heavy chain region sequence shares 100% sequence identity or sequence similarity with the sequence of germline DP50 or with the light chain sequence of germline A27, or germline O12.

In one embodiment of the present invention, the antibody shares at least 80%, more preferably, at least 85%, even more preferably, at least 90%, yet more preferably, at least 95%, more preferably, at least 99%, sequence identity or sequence similarity with the heavy chain, the light chain, or both, sequences from FR1 through FR4 with the FR1 through FR4 region sequences of antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab. Even more preferably, the antibody shares 100% sequence identity or sequence similarity over the heavy, light, or both, sequences from FR1 through FR4 with antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, and ipilimumab.

In another embodiment of the present invention, the antibody shares at least 80%, more preferably, at least 85%, even more preferably, at least 90%, yet more preferably, at least 95%, more preferably, at least 99%, and most preferably, about 100%, sequence identity or sequence similarity with the heavy chain sequences from FR1 through FR3 with the FR1 through FR3 region sequences of germline DP50.

In yet another embodiment of the present invention, the antibody shares at least 80%, more preferably, at least 85%, even more preferably, at least 90%, yet more preferably, at least 95%, more preferably, at least 99%, and most preferably, about 100%, sequence identity or sequence similarity with the light chain sequences from FR1 through FR4 with the FR1 through FR4 region sequences of germline V_{κ} A27, or germline V_{κ} O12.

In one embodiment of the present invention, the antibody shares at least 80%, more preferably, at least 85%, even more preferably, at least 90%, yet more preferably, at least 95%, more preferably, at least 99%, sequence identity or sequence similarity with the heavy chain, the light chain, or both, CDR-1, CDR-2 and CDR-3 sequences of antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, ipilimumab. Even more preferably, the antibody shares 100% sequence identity or sequence similarity over the heavy, light, or both, CDR-1, CDR-2 and CDR-3 sequences with antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, and ipilimumab.

In another embodiment of the present invention, the antibody shares at least 80%, more preferably, at least 85%, even more preferably, at least 90%, yet more preferably, at least 95%, more preferably, at least 99%, and most preferably, about 100%, sequence identity or sequence similarity with the heavy chain CDR-1 and CDR-2 sequences with the CDR-1 and CDR-2 sequences of germline DP50.

In yet another embodiment of the present invention, the antibody shares at least 80%, more preferably, at least 85%, even more preferably, at least 90%, yet more preferably, at least 95%, more preferably, at least 99%, and most preferably, about 100%, sequence identity or sequence similarity with the light chain CDR-1, CDR-2 and CDR-3 sequences with the CDR-1, CDR-2 and CDR-3 sequences of germline V_{κ} A27, or germline V_{κ} O12.

Examples of antibodies employable in the present invention, and methods of producing them, are described in, among others, U.S. patent application Ser. No. 09/472,087, now issued as U.S. Pat. No. 6,682,736; Int. Appl. No. PCT/US00/23356 (published Mar. 1, 2001, as WO 01/14424) (e.g., antibody ipilimumab, also known as MDX-010, Medarex, Princeton, N.J.); Int. Appl. No. PCT/US99128739 (published Jun. 8, 2000, as WO 00/32231); U.S. Pat. Nos. 5,811,097, 5,855,887, 6,051,227, and 6,207,156; each of which is incorporated by reference herein. While information on the amino and nucleic acid sequences relating to these antibodies is provided herein, further information can be found in U.S. Pat. No. 6,682,736, as well as WO 00/37504; the sequences set forth in those applications are hereby incorporated herein by reference.

Certain uses for these antibodies to treat various cancers were discussed in U.S. patent application Ser. No. 10/153,382, now published as U.S. Patent Application Publication No. 2003/0086930, which is incorporated by reference as if set forth in its entirety herein.

Characteristics of human anti-CTLA4 antibodies useful in the methods of the invention are extensively discussed in, e.g., U.S. Pat. No. 6,682,736, and include antibodies having amino acid sequences of an antibody such as, but not limited to, antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, and ipilimumab. The invention also relates to methods using antibodies comprising the amino acid sequences of the CDRs of the heavy and light chains of these antibodies, as well as those comprising changes in the CDR regions, as described in the above-cited applications and patent. The invention also concerns antibodies comprising the variable regions of the heavy and light chains of those antibodies. In another embodiment, the antibody is selected from an antibody comprising the full length, variable region, or CDR, amino acid sequences of the heavy and light chains of antibodies 3.1.1, 4.1.1, 4.8.1, 4.10.2, 4.13.1, 4.14.3, 6.1.1, ticilimumab, 11.6.1, 11.7.1, 12.3.1.1, and 12.9.1.1, and ipilimumab.

While the anti-CTLA4 antibodies discussed previously herein may be preferred, the skilled artisan, based upon the disclosure provided herein, would appreciate that the invention encompasses a wide variety of anti-CTLA4 antibodies and is not limited to these particular antibodies. More particularly, while human antibodies are preferred, the invention is in no way limited to human antibodies; rather, the invention encompasses useful antibodies regardless of species origin, and includes, among others, chimeric, humanized and/or primatized antibodies. Also, although the antibodies exemplified herein were obtained using a transgenic mammal, e.g., a mouse comprising a human immune repertoire, the skilled artisan, based upon the disclosure provided herein, would understand that the present invention is not limited to an antibody produced by this or by any other particular method. Instead, the invention includes an anti-CTLA4 antibody produced by any method, including, but not limited to, a method known in the art (e.g., screening phage display libraries, and the like) or to be developed in the future for producing an anti-CTLA4 antibody of the invention. Based upon the extensive disclosure provided herein and in, e.g., U.S. Pat. No. 6,682,736, to Hanson et al., and U.S. Pat. App. Pub. No. 2002/0088014, one skilled in the art can readily produce and identify an antibody useful for treatment of prostate cancer in combination with a hormonal therapeutic agent using the novel methods disclosed herein.

The present invention encompasses human antibodies produced using a transgenic non-human mammal, i.e., XenoMouse™ (Abgenix, Inc., Fremont, Calif.) as disclosed in the U.S. Pat. No. 6,682,736, to Hanson et al.

Another transgenic mouse system for production of “human” antibodies is referred to as “HuMAb-Mouse™” (Medarex, Princeton, N.J.), which contains human immunoglobulin gene miniloci that encodes unrearranged human heavy (mu and gamma) and kappa light chain immunoglobulin sequences, together with targeted mutations that inactivate the endogenous mu and kappa chain loci (Lonberg et al. Nature 368:856-859 (1994), and U.S. Pat. No. 5,770,429).

However, the invention uses human anti-CTLA4 antibodies produced using any transgenic mammal such as, but not limited to, the Kirin TC Mouse™ (Kirin Beer Kabushiki Kaisha, Tokyo, Japan) as described in, e.g., Tomizuka et al., Proc Natl Acad Sci USA 97:722 (2000); Kuroiwa et al., Nature Biotechnol 18:1086 (2000); U.S. Patent Application Publication No. 2004/0120948, to Mikayama et al.; and the HuMAb-Mouse™ (Medarex, Princeton, N.J.) and XenoMouse™ (Abgenix, Inc., Fremont, Calif.), supra. Thus, the invention encompasses using an anti-CTLA4 antibody produced using any transgenic or other non-human animal.

In another embodiment, the antibodies employed in methods of the invention are not fully human, but “humanized”. In particular, murine antibodies or antibodies from other species can be “humanized” or “primatized” using techniques well known in the art. See, e.g., Winter and Harris Immunol. Today 14:43-46 (1993), Wright et al. Crit. Reviews in Immunol. 12:125-168 (1992), and U.S. Pat. No. 4,816,567, to Cabilly et al, and Mage and Lamoyi in Monoclonal Antibody Production Techniques and Applications pp. 79-97, Marcel Dekker, Inc., New York, N.Y. (1987). Thus, humanized, chimeric antibodies, anti-CTLA4 antibodies derived from any species (including single chain antibodies obtained from camelids as described in, e.g., U.S. Pat. Nos. 5,759,808 and 6,765,087, to Casterman and Hamers), as well as any human antibody, can be combined with a therapeutic agent to practice the novel methods disclosed herein.

As will be appreciated based upon the disclosure provided herein, antibodies for use in the invention can be obtained from a transgenic non-human mammal, and hybridomas derived therefrom, but can also be expressed in cell lines other than hybridomas.

Mammalian cell lines available as hosts for expression are well known in the art and include many immortalized cell lines available from the American Type Culture Collection (ATCC), including but not limited to Chinese hamster ovary (CHO) cells, NS0, Sp2, HEK, HeLa cells, baby hamster kidney (BHK) cells, monkey kidney cells (COS), and human hepatocellular carcinoma cells (e.g., Hep G2). Non-mammalian prokaryotic and eukaryotic cells can also be employed, including bacterial, yeast, insect, and plant cells.

Various expression systems can be used as well known in the art, such as, but not limited to, those described in, e.g., Sambrook and Russell, Molecular Cloning, A Laboratory Approach, Cold Spring Harbor Press, Cold Spring Harbor, N.Y. (2001), and Ausubel et al., Current Protocols in Molecular Biology, John Wiley & Sons, NY (2002). These expression systems include dihydrofolate reductase (DHFR)-based systems, among many others. The glutamine synthetase system of expression is discussed in whole or part in connection with European Patents No. 0216846B1, No. 0256055B1, and No. 0323997B1, and European Patent Application No. EP89303964. In one embodiment, the antibody used is made in NS0 cells using a glutamine synthetase system (GS-NS0). In another embodiment, the antibody is made in CHO cells using a DHFR system. Both systems are well-known in the art and are described in, among others, Barnes et al. Biotech & Bioengineering 73:261-270 (2001), and references cited therein.

Site directed mutagenesis of the antibody CH2 domain to eliminate glycosylation may be preferred in order to prevent changes in either the immunogenicity, pharmacokinetic, and/or effector functions resulting from non-human glycosylation. Further, the antibody can be deglycosylated by enzymatic (see, e.g., Thotakura et al. Meth. Enzymol. 138:350 (1987)) and/or chemical methods (see, e.g., Hakimuddin et al., Arch. Biochem. Biophys. 259:52 (1987)).

Further, the invention encompasses using an anti-CTLA4 antibody comprising an altered glycosylation pattern. The skilled artisan would appreciate, based upon the disclosure provided herein, that an anti-CTLA4 antibody can be modified to comprise additional, fewer, or different glycosylations sites compared with the naturally-occurring antibody. Such modifications are described in, e.g., U.S. Patent Application Publication Nos. 2003/0207336, and 2003/0157108, and International Patent Publication Nos. WO 01/81405 and 00/24893.

Additionally, the invention comprises using an anti-CTLA4 antibody regardless of the glycoform, if any, present on the antibody. Moreover, methods for extensively remodeling the glycoform present on a glycoprotein are well-known in the art and include, e.g., those described in International Patent Publication Nos. WO 03/031464, WO 98/58964, and WO 99/22764, and US Patent Application Publication Nos. 2004/0063911, 2004/0132640, 2004/0142856, 2004/0072290, and U.S. Pat. No. 6,602,684 to Umana et al.

Further, the invention encompasses using an anti-CTLA4 antibody with any art-known covalent and non-covalent modification, including, but not limited to, linking the polypeptide to one of a variety of nonproteinaceous polymers, e.g., polyethylene glycol, polypropylene glycol, or polyoxyalkylenes, in the manner set forth in, for example, U.S. Patent Application Publication Nos. 2003/0207346 and 2004/0132640, and U.S. Pat. Nos. 4,640,835; 4,496,689; 4,301,144; 4,670,417; 4,791,192; 4,179,337.

Additionally, the invention encompasses using an anti-CTLA4 antibody, or antigen-binding portion thereof, chimeric protein comprising, e.g., a human serum albumin polypeptide, or fragment thereof. Whether the chimeric protein is produced using recombinant methods by, e.g., cloning of a chimeric nucleic acid encoding the chimeric protein, or by chemical linkage of the two peptide portions, the skilled artisan would understand once armed with the teachings provided herein that such chimeric proteins are well-known in the art and can confer desirable biological properties such as, but not limited to, increased stability and serum half-life to the antibody of the invention and such molecules are therefore included herein.

Antibodies that are generated for use in the invention need not initially possess a particular desired isotype. Rather, the antibody as generated can possess any isotype and can be isotype switched thereafter using conventional techniques. These include direct recombinant techniques (see, e.g., U.S. Pat. No. 4,816,397), and cell-cell fusion techniques (see e.g., U.S. Pat. No. 5,916,771.

The effector function of the antibodies of the invention may be changed by isotype switching to an IgG1, IgG2, IgG3, IgG4, IgD, IgA, IgE, or IgM for various therapeutic uses. Furthermore, dependence on complement for cell killing can be avoided through the use of bispecifics, immunotoxins, or radiolabels, for example.

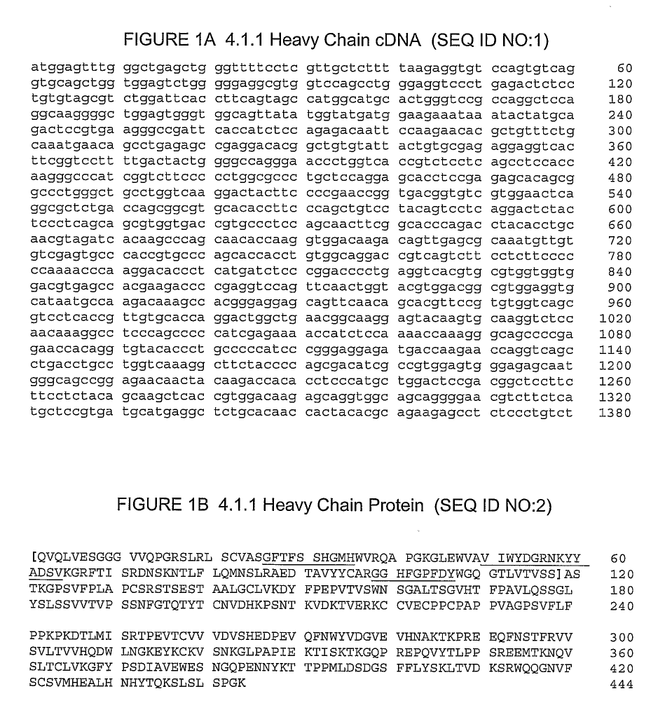

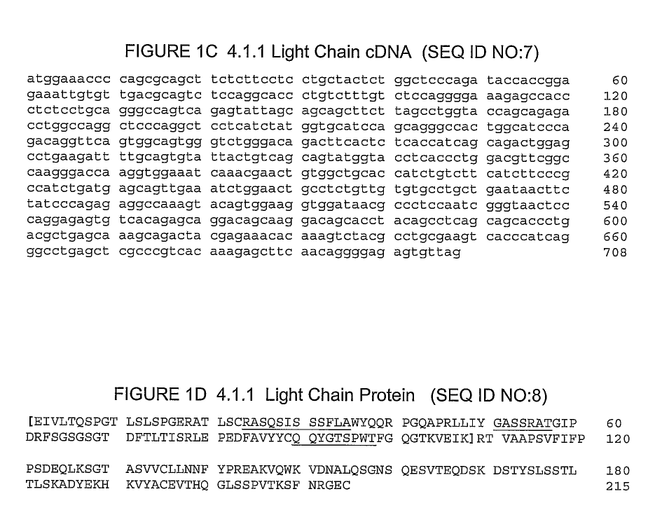

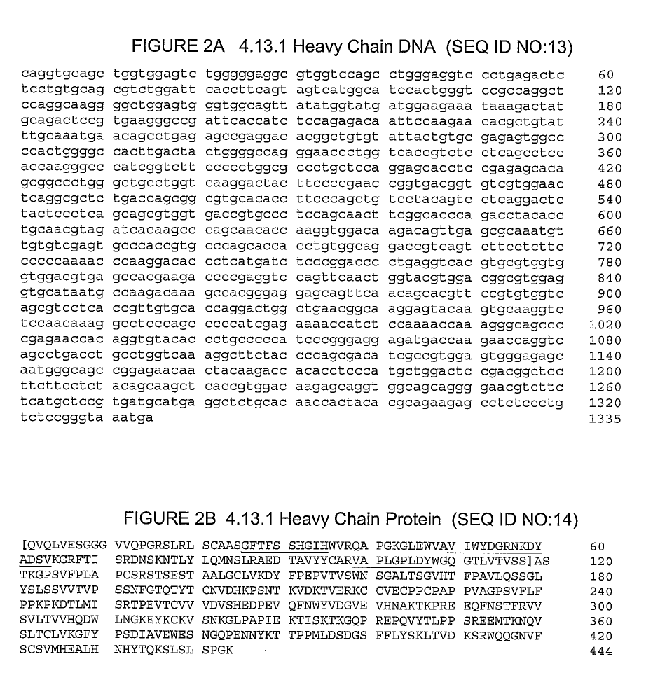

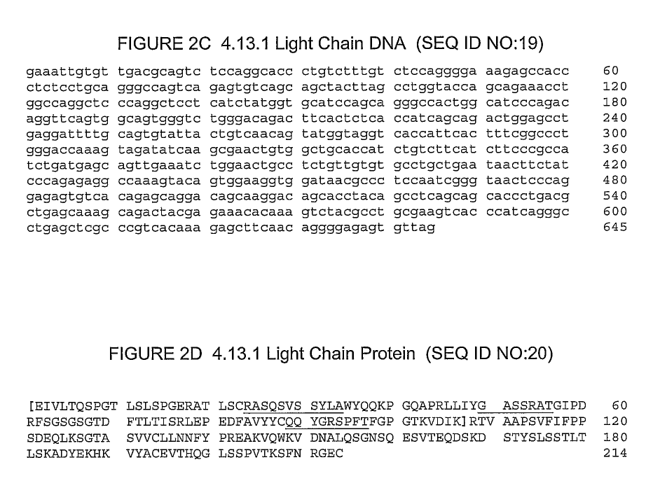

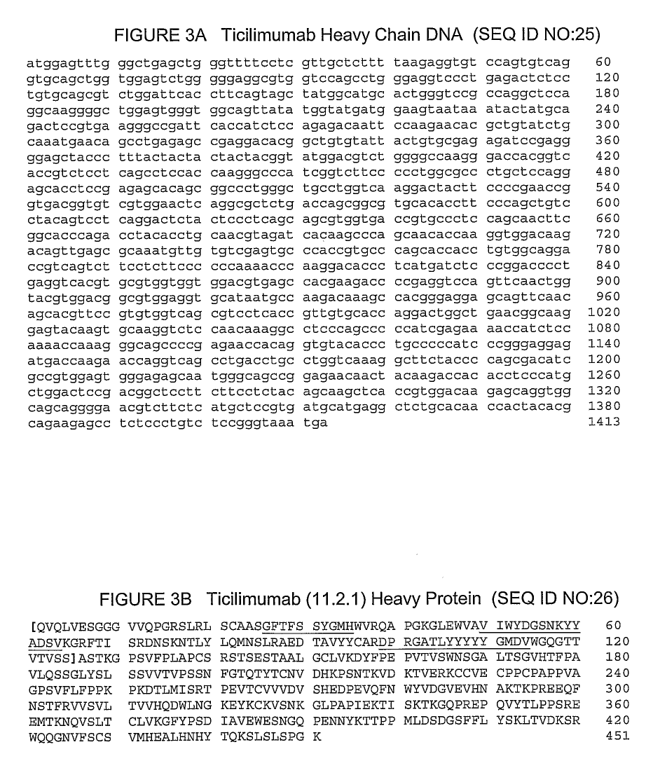

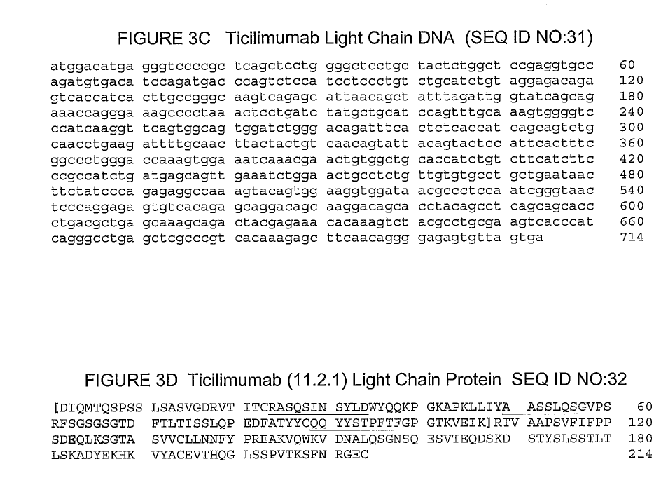

Although antibody 4.1.1, 4.13.1 and ticilimumab are IgG2 antibodies and the sequences of the variable regions of the antibodies are provided herein (FIGS. 1-3), and in the applications and patents referenced and incorporated herein, it is understood that the full-length sequences of these antibodies are encompassed herein, as well as the use of any antibody comprising the sequences set forth in SEQ ID NOs:1-36, and further comprising any constant region, regardless of isotype as more fully discussed elsewhere herein. Likewise, any antibody comprising the full-length sequence of ipilimumab, or any portion thereof, including a sequence encoding an antigen-binding portion of ipilimumab, can be used according to the methods of the invention.

Thus, the skilled artisan, once provided with the teachings provided herein, would readily appreciate that the anti-CTLA4 antibody-therapeutic agent combination of the invention can comprise a wide plethora of anti-CTLA4 antibodies.

Further, one skilled in the art, based upon the disclosure provided herein, would understand that the invention is not limited to administration of only a single antibody; rather, the invention encompasses administering at least one anti-CTLA4 antibody, e.g., one of 4.1.1, 4.13.1, or ticilimumab, in combination with a therapeutic agent. Further, any combination of anti-CTLA-4 antibodies can be combined with at least one therapeutic agent and the present invention encompasses any such combination and permutation thereof.

In one embodiment of the invention, the methods use the anti-CTLA4 antibody designated ticilimumab. In another embodiment of the invention, the methods use an anti-CTLA4 antibody described in, e.g., the following applications and patents: U.S. patent application Ser. No. 09/472,087, now issued as U.S. Pat. No. 6,682,736; Int. Appl. No. PCT/US99/30895 (published Jun. 29, 2000, as WO 00/37504); U.S. patent application Ser. No. 10/612,497 (published Nov. 18, 2004, as US 2004/0228858); U.S. patent application Ser. No. 10/776,649 (published Nov. 18, 2004, as US 2004/0228861); Int. Appl. No. Int. Appl. No. PCT/US00/23356 (published Mar. 1, 2001, as WO 01/14424) (e.g., antibody 10D1, also known as MDX-010, and ipilimumab, Medarex, Princeton, N.J.); Int. Appl. No. PCT/US99/28739 (published Jun. 8, 2000, as WO 00/32231); U.S. Pat. Nos. 5,811,097, 5,855,887, 6,051,227, and 6,207,156; U.S. Pat. No. 5,844,095, to Linsley et al. Int. Appl. No. PCT/US92/05202 (published Jan. 7, 1993, as WO 93/00431); U.S. patent application Ser. No. 10/153,382 (published May 8, 2003, as US 2003/0086930); U.S. patent application Ser. No. 10/673,738 (published Feb. 24, 2005 as US 2005/0042223); U.S. patent application Ser. No. 11/085,368 (published Oct. 13, 2005, as US 2005/0226875); U.S. Pat. Appl. No. 60/624,856 (filed Nov. 4, 2004); U.S. Pat. Appl. No. 60/664,364 (filed Mar. 23, 2005); U.S. Pat. Appl. No. 60/664,653 (filed Mar. 23, 2005); U.S. Pat. Appl. No. 60/697,082 (filed Jul. 7, 2005); U.S. Pat. Appl. No. 60/711,707 (filed Aug. 26, 2005).

III. Dosage Regimens

Dosage regimens may be adjusted to provide the optimum desired response for the relevant method of the invention. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. “Dosage unit form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of individuals.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

An exemplary, non-limiting range for a therapeutically effective amount of an antibody administered according to the invention is at least about 1 mg/kg, at least about 5 mg/kg, at least about 10 mg/kg, more than about 10 mg/kg, or at least about 15 mg/kg, for example about 1-30 mg/kg, or for example about 1-25 mg/kg, or for example about 1-20 mg/kg, or for example about 5-20 mg/kg, or for example about 10-20 mg/kg, or for example about 15-20 mg/kg, or for example, about 15 mg/kg. In one embodiment, the amount is 10 mg/kg. In another, it is 15 mg/kg.

Further, dose escalation protocol may be used to determine the maximum tolerated dose (MTD), to assess dose limiting toxicity (DLT), if any, associated with administration of antibody-hormonal therapy combination therapy. Dose escalation comprises increasing doses, such as, but not limited to, 0.1 mg/kg, 0.3 mg/kg, 1 mg/kg, 3, mg/kg, 6 mg/kg, 10 mg/kg, and 15 mg/kg, or any combination thereof. In another embodiment, successive doses of 3 mg/kg, 6 mg/kg and 10 mg/kg are administered and the patient is assessed for toxicity, if any, as well as for efficacy of treatment, among other parameters.

In one embodiment, the antibody is administered in an intravenous formulation as a sterile aqueous solution containing about 5 to 20 mg/ml of antibody, in an appropriate buffer system.

In one embodiment, part of the dose is administered by an intraveneous bolus and the rest by infusion of the antibody formulation. For example, a 0.01 mg/kg intravenous injection of the antibody may be given as a bolus, and the rest of a predetermined antibody dose may be administered by intravenous injection. A predetermined dose of the antibody may be administered, for example, over a period of about an hour and a half to about five hours. In one embodiment, the antibody is administered as a single IV infusion at about 100 ml per hour, more preferably, the rate is about 200 ml per hour, and the infusion rate may be determined by the skilled artisan according to art-recognized methods.

In one embodiment, a single dose or multiples doses of the antibody may be administered. The anti-CTLA4 antibody may be administered to a human as frequently as several times daily, or it may be administered less frequently, such as once a day, once a week, once every two weeks, once a month, or even less frequently, such as once every several months or even once a year or less. The doses may be administered, for example, every two weeks, monthly, every twenty days, every 25 days, every 28 days, every 30 days, every 40 days, every 50 days, every two months, every 70 days, every 80 days, every three months, every six months or yearly. In addition, the hormonal therapy agent can be administered several times per day, once per day, several times per week, weekly, every other week, every third week, every fourth week, monthly, every three months, every six months, once per year, or any other period that provides a therapeutic benefit to the patient. The frequency of the dose will be readily apparent to the skilled artisan and will depend upon any number of factors, such as, but not limited to, the type and severity of the disease being treated, and age of the human, etc.

In one embodiment of the invention where administration of the antibody and hormone therapy overlap, the antibody and hormonal therapy agent can be co-administered in that they can be administered separately, or at different times of the day, as well as simultaneously or on the same date, such that co-administration is substantially contemporaneous. Co-administration thus encompasses substantially contemporaneous administration of the antibody and the hormonal therapy agent such that administration of the two mediates a therapeutic benefit to the patient that is detectably greater than administration of either agent in the absence of the other.

Additionally, in certain embodiments of the invention, the antibody and hormonal therapy agent are administered where the hormonal therapy agent is administered prior to administration of the antibody and where there is resting period between administration of the last dose of hormonal therapy agent and administration of the antibody. The duration of the resting phase can be adjusted according to a variety of factors well-known in the art.

As stated previously, in certain embodiments, the antibody and hormone therapy agent combination is further combined with numerous additional compounds (other therapeutic agents, cytokines, chemotherapeutic and/or antiviral drugs, among many others). Alternatively, the compound(s) may be administered an hour, a day, a week, a month, or even more, in advance of the antibody-therapeutic agent combination, or any permutation thereof. Further, the compound(s) may be administered an hour, a day, a week, or even more, after administration of radiation, stem cell transplant, or administration of any therapeutic agent (e.g., cytokine, chemotherapeutic compound, and the like), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds, and the like.

Leuprolide is preferably administered subcutaneously (s.c.) or intramuscularly (i.m.; LUPRON DEPOT) according to standard dosing regimens well-known in the art comprising, inter alia, administering the compound daily, monthly, every other month, at three or four month intervals, and the like. Moreover, the dose of leuprolide/LUPRON DEPOT can be adjusted depending on various factors as understood by the skilled artisan, and can conventionally range from about 1 mg daily for leuprolide administered s.c., to about 3.5 and 7.5 mg per dose for LUPRON DEPOT administered monthly i.m., and 11.23 and 22.5 mg for doses of LUPRON DEPOT administered every three months, and about 30 mg for LUPRON DEPOT administered every four months. Alternative regimens encompass, inter alia, 1 mg leuprolide administered daily as a single subcutaneous injection, 22.5 mg administered every three months as one intramuscular injection, among many other regimens known in the art. In certain embodiments of the invention, the anti-CTLA4 antibody is administered at any time during, prior or following administration of leuprolide. Preferably, leuprolide is administered intramuscularly every twenty-eight days, concurrent with intravenous administration of the anti-CTLA4 antibody. Even more preferably, leuprolide is administered in the amount of approximately 7.5 mg intramuscularly.

In embodiments of the invention where administration of leuprolide and antibody overlap, in particular where the antibody is administered with leuprolide and bicalumatide, three cycles of leuprolide/antibody can be administered every twenty-eight days. Optionally, at least one additional cycle of leuprolide/antibody is administered following prostatectomy. More preferably, about three cycles of leuprolide/antibody are administered post-surgery. Even more preferably, the first post-surgery cycle commences within forty-five days following surgery to maintain a therapeutic level of antibody present in the patient.

Bicalutamide can be administered according to standard dosing regimens well-known in the art. Briefly, bicalutamide is preferably administered once per day in an amount ranging from about 50 mg to 200 mg. Preferably, bicalutamide is administered per os (by mouth) in an amount of approximately 50 mg per day. Even more preferably, bicalutamide is administered for the first fourteen days of the first dosing cycle, yet more preferably, bicalutamide, leuprolide and the anti-CTLA4 are co-administered on the first day of the first cycle (D1). Thereafter, bicalutamide is preferably discontinued after day 14 (D14). In one embodiment, leuprolide and the antibody are co-administered on D28 and D56. Even more preferably, leuprolide is administered by i.m. injection at about 7.5 mg and the antibody is administered by i.v. infusion at a dose ranging from about 1 mg/kg to 20 mg/kg, in another embodiment, from about 3 mg/kg to 15 mg/kg, and in another embodiment, from about 3 mg/kg to 10 mg/kg.

The skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined. Accordingly, while certain dose and administration regimens are exemplified herein, these examples in no way limit the dose and administration regimen that can be provided to a patient in practicing the present invention. Further, one skilled in the art would understand, once armed with the teachings provided herein, that a therapeutic benefit, such as, but not limited to, detectable decrease in tumor size and/or metastasis, decreased level of PSA, increased time to recurrence, among many other parameters, can be assessed by a wide variety of methods known in the art for assessing the efficacy of treatment of prostate cancer, and these methods are encompassed herein, as well as methods to be developed in the future.

In one embodiment, a single bolus injection comprising the anti-CTLA4 antibody is administered to a patient intravenously at a dose ranging from about 1 mg/kg to 20 mg/kg approximately every twenty-eight days. In another embodiment of the invention, ticilimumab is administered at a dose of about 10 mg/kg every twenty-eight days. In yet another embodiment, ticilimumab is administered at about 15 mg/kg every three months. A dose of a LH-RH agonist (leuprolide) is administered on that first day that the antibody is administered, and approximately every twenty-eight days thereafter. Preferably, the antibody and leuprolide are co-administered on the same day of each dose cycle. In another embodiment, leuprolide is administered every twenty-eight days and ticilimumab is administered every three months. Additionally, bicalutamide is administered daily for fourteen days starting on the day the antibody and leuprolide are co-administered. Preferably, bicalutamide is not administered during subsequent dose cycles; however, bicalutamide may be administered in subsequent cycles and/or for longer than fourteen days. Further, the invention encompasses administering bicalutamide and/or leuprolide at any point during administration of the antibody and the invention is not limited in any way with respect to the relative administration of the antibody and the hormonal therapy agent. Thus, hormonal therapy can be administered either before, during and/or after administration of the antibody.

IV. Pharmaceutical Compositions

The invention encompasses the preparation and use of pharmaceutical compositions comprising an anti-CTLA4 antibody, or an antigen-binding portion thereof, in combination with at least two hormonal therapy agents independently selected from, e.g., an anti-androgen, a GnRH antagonist, and a LH-RH agonist. Such a pharmaceutical composition may consist of each of an antibody or a hormonal therapy agent alone (e.g., an effective dose of an anti-CTLA4, an effective dose of at least one hormonal therapy agent) in a form suitable for administration to a subject, or the pharmaceutical composition may comprise the antibody or hormonal therapy agent and one or more pharmaceutically acceptable carriers, one or more additional (active and/or inactive) ingredients, or some combination of these.

In one embodiment, the antibody is administered parenterally (e.g., intravenously) in an aqueous solution while the hormonal therapy agent (e.g., an anti-androgen, a GnRH antagonist, a LH-RH agonist, and the like) is administered orally in pill/capsule form. However, the skilled artisan would understand, based upon the disclosure provided herein, that the invention is not limited to these, or any other, formulations, doses, routes of administration, and the like. Rather, the invention encompasses any formulation or method of administering an antibody in combination with a hormonal therapy agent, including, but not limited to, administering each agent separately in a different formulation via a different route of administration (e.g., administering an anti-CTLA4 antibody i.v., while co-administering a non-steroidal anti-androgen (bicalutamide) orally and a LH-RH agonist (leuprolide) by intramuscular injection), and administering the antibody and hormonal therapy (e.g., a second anti-CTLA4 antibody, such as, but not limited to, ipilimumab, among others) in a single composition (e.g., in an aqueous composition administered, inter alia, i.v.), among many others. Thus, the following discussion describes various formulations for practicing the methods of the invention comprising administration of any anti-CTLA4 antibody in combination with any hormonal therapy agent, but the invention is not limited to these formulations, but comprises any formulation as can be readily determined by one skilled in the art once armed with the teachings provided herein for use in the methods of the invention.

The antibodies employed in the invention can be incorporated into pharmaceutical compositions suitable for administration to a subject. Typically, the pharmaceutical composition comprises the antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, trehalose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The antibodies may be in a variety of forms. These include, for example, liquid forms, such as liquid solutions (e.g., injectable and infusible solutions), dispersions or suspensions, and liposomes. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies can be administered by a variety of methods known in the art, including, without limitation, oral, parenteral, mucosal, inhalation, topical, buccal, nasal, and rectal. For many therapeutic applications, the preferred route/mode of administration is subcutaneous, intramuscular, intravenous or infusion. Non-needle injection may be employed, if desired. As will be appreciated by the skilled artisan, the route and/or mode of administration will vary depending upon the desired results.

In certain embodiments, the antibody may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York (1978).

In one embodiment, the antibody is administered in an intravenous formulation as a sterile aqueous solution containing 5 or 10 mg/ml of antibody, with sodium acetate, polysorbate 80, and sodium chloride at a pH ranging from about 5 to 6. Preferably, the intravenous formulation is a sterile aqueous solution containing 5 or 10 mg/ml of antibody, with 20 mM sodium acetate, 0.2 mg/ml polysorbate 80, and 140 mM sodium chloride at pH 5.5.

In another embodiment of the invention, the antibody is administered in a sterile solution comprising 20 mM histidine buffer, pH 5.5, 84 mg/ml trehalose dihydrate, 0.2 mg/ml polysorbate 80, and 0.1 mg/ml disodium EDTA dihydrate. In one aspect, the formulation is packaged in clear glass vials with a rubber stopper and an aluminum seal. In another aspect, the vial contains about 20 mg/ml of antibody with a nominal fill of about 400 mg per vial.

With regard to a hormonal therapy agent, the agent can be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics, anti-diarrheals, chemotherapeutic agents, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

V. Kits

The invention includes various kits for treatment of prostate cancer. In one embodiment, the kit is used for treatment of hormone-dependent prostate cancer and comprises a therapeutically effective amount of an anti-CTLA4 antibody, or an antigen-binding portion thereof, and a therapeutically effective amount of at least two hormonal therapy agents, along with an applicator and instructional materials which describe use of the combination to perform the methods of the invention. Although exemplary kits are described below, the contents of other useful kits will be apparent to the skilled artisan in light of the present disclosure. Each of these kits is included within the invention.

The invention includes a kit for treatment of hormone-independent prostate cancer in a patient in need thereof. The kit includes a human anti-CTLA4 antibody of the invention and at least one hormonal therapy agent. The kit further comprises an applicator, including, but not limited to, a syringe, for administration of the components of the kit to a patient. Further, the kit comprises an instructional material setting forth the pertinent information for the use of the kit to treat prostate cancer in the patient.

In one embodiment, the kit comprises at least one anti-CTLA4 antibody selected from 4.1.1, 4.8.1, 4.10.2, 4.13.1, 4.14.3, 6.1.1, ticilimumab, 11.6.1, 11.7.1., 12.3.1.1, 12.9.1.1, and 10D1 (ipilimumab; MDX-010, Medarex), even more preferably, the antibody is selected from 4.1.1, 4.13.1, 6.1.1, ticilimumab, and ipilimumab. More preferably, the antibody is selected from 4.1.1, 4.13.1, 4.14.3, 6.1.1, and ticilimumab.

In one embodiment, the hormonal therapy agent is at least one agent selected from an anti-androgen, a GnRH antagonist, and a LH-RH agonist, among others. In one aspect, the anti-androgen is either a steroidal or non-steroidal anti-androgen. In another aspect, the non-steroidal anti-androgen is selected from bicalutamide, nilutamide, flutamide, among others. A steroidal anti-androgen is selected from the group consisting of megestrol, cyproterone, and the like. In yet another aspect, the GnRH is selected from the group consisting of abarelix and histrelin. And in a further aspect, the LH-RH is selected from the group consisting of leuprolide, goserelin, buserelin, and tryptorelin, and the like.

The invention encompasses a kit for treatment of hormone-independent prostate cancer, where the kit comprises any combination of an anti-CTLA4 antibody and any hormonal agent, such as, but not limited to, leuprolide, bicalutamide, and an antibody. While such kit is preferred, the invention is not limited to this particular combination. Rather, the kit can comprise any combination of known hormonal therapy agents known to reduce androgen levels. Further, the kit can comprise a wide plethora of additional agents for treatment of cancer. Such agents are set forth previously and include chemotherapeutic compounds, cancer vaccines, signal transduction inhibitors, agents useful in treating abnormal cell growth or cancer, antibodies or other ligands that inhibit tumor growth by binding to IGF-1R, a chemotherapeutic agent (taxane, vinca alkaloid, platinum compound, intercalating antibiotics, among many others), and cytokines, among many others, as well as palliative agents to treat, e.g., any toxicities that arise during treatment.

The disclosures of each and every patent, patent application, and publication cited herein are hereby incorporated herein by reference in their entirety.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Anti-CTLA4 in Combination with at Least Two Hormonal Therapy Agents in the Neoadjuvant Treatment of High Risk Prostate Cancer

Patients with prostate cancer who are eligible for radical prostatectomy and who have an intermediate or high risk of recurrence according to current medical standards are candidates for antibody-hormonal combination neoadjuvant therapy. The target population for this study is defined using the risk of biochemical recurrence instead of the probability of pathologically confined cancer. This approach is supported by the observation that patients with disease confined to the prostate may have a biochemical recurrence despite “definitive” local treatment. Therefore, this patient definition is more comprehensive. Considering recent data showing that a time to PSA failure of less than 2 years predicts for distant failure, patients who are at high risk for early PSA failure are candidates for neoadjuvant (and adjuvant) antibody-hormonal combination therapy as disclosed herein.

For all patients, ECOG (Eastern Cooperative Oncology Group) performance status, vital signs, and body weight are assessed pre-dose, and vital signs are repeated post-dose, as clinically indicated. A physical examination (including opthalmologic assessment and signs of autoimmunity) is performed on Day 1. Samples for hematology panel (hematocrit, RBC count, WBC count, differential), chemistry panel (Alkaline Phosphatase, calcium, chloride, GGT, LDH, magnesium, phosphorus, random glucose, sodium, urea, uric acid), urinalysis (including proteinuria and urine sediment), and others (activated partial thromboplastin time [APTT], prothrombin time (PT), autoantibody panel, C reactive protein, TSH, T3, T4, amylase, lipase, serum C3, C4, serum Ig level, PSA level), are obtained.

Baseline human anti-human antibody (HAHA) titer is determined and pharmacokinetic (PK) specimen is obtained pre-dose. Pharmacokinetics are obtained at 0 hour (just prior to dosing) and at 1 hour after the antibody infusion.

At day 14, ECOG PS and adverse invents, if any, are evaluated. Samples for hematology, chemistry labs, along with PT, APTT, CRP, and PSA are obtained.

Pre-operatively, approximately after the end of the last cycle of antibody-hormone combination therapy and prior to scheduled prostatectomy, the following evaluations are completed: any adverse events, ECOG PS, vital signs and weight, physical examination (including signs of autoimmunity), ECG, and DRE. Samples for hematology and chemistry panels, along with PT, APTT, CRP and PSA are obtained.

Post-operative assessments are performed approximately two weeks following surgery. The evaluation includes physical examination (including autoimmunity), vital signs, ECOG performance status, body weight, hematology, and chemistry panels, PT, APTT, TSH, T3, T4, amylase, lipase, CRP, urinalysis. Additional samples are obtained for autoantibody panel, PSA, testosterone, HAH titer and PK. All patients are assessed for pathological assessment of the surgical specimen and adverse events.

For patients who do not receive additional cycles of antibody-hormonal combination therapy, the patients are evaluated approximately one month after the post-operative assessment. Patients are assessed for ECOG PS, a physical examination (including signs of autoimmunity), and a digital rectal exam. A blood sample for PSA is also obtained.

Patients are followed every three months until disease recurrence or PSA progression or a maximum of 24 months or until the start of an alternative therapeutic regimen, whichever comes first.

Patients who have demonstrated a pathological response, optionally receive up to three additional cycles of antibody without hormone therapy every twenty-eight days. Preferably, the antibody is administered to the patients within 45 days following surgery.

Patients are evaluated at days 1, 14 and for follow-up as described previously for the cycles of antibody-hormonal combination therapy.

The following endpoints are measured: PK parameters, HAHA, response rate and time to progression. Time to progression and overall survival are calculated using the Kaplan-Meier product limit method.

The antibody is provided in 10 ml clear glass vials with a rubber stopper and an aluminum seal. Each vial contains 5 mg/ml (with a nominal fill of 50 mg/vial) of anti-CTLA4 antibody, in a sterile aqueous solution comprising 20 mM sodium acetate, 0.2 mg/ml polysorbate 80, and 140 mM sodium chloride at pH 5.5.

The patient is premedicated with antihistamine (H1) at least one half hour prior to infusion of anti-CTLA4, but premedication is not required. Patients were administered leuprolide (LUPRON) and bicalutamide (CASODEX) in combination with an anti-CTLA4 antibody (ticilimumab). More specifically, on day 1 (D1), the patient was administered a single IV infusion (100 mL/hr) of anti-CTLA4 antibody at a dose of 1 mg/kg, 3 mg/kg, or 6 mg/kg, in combination with a 7.5 mg IM injection of leuprolide (LUPRON DEPOT). A minimum of 3 patients were administered each antibody dose. The study was amended to reinitiate dose escalation at a lower dose level, i.e., 1 mg/kg. Three patients received that dose level, and are undergoing surgery (after 3 months of combination therapy). Dose escalation continued to 3 mg/kg. Dose escalation is ongoing at 6 mg/kg and 10 mg/kg.

Bicalutamide (CASODEX) was administered orally, in tablet form, at a dose of 50 mg, once per day for the first fourteen days commencing on day 1 (D1). Preferably, bicalutamide was taken at the same time each day on days D1-D14, and a patient medication diary was used to record any interruption of bicalutamide dosing.

Prophylactic anti-emetics and anti-diarrheals were given as appropriate. The treatment was repeated after 28 days with a transfusion of antibody and 7.5 mg IM injection of LUPRON DEPOT for a maximum of three cycles (D1, D28, and D56) preceding surgery.

Optionally, the antibody dose is escalated, e.g., from 1 to 3 mg/kg, from 3 to 6 mg/kg, and from 6 to 10 mg/kg, and the like. Doses are escalated using an accelerated titration design utilizing a dose-doubling schema with 3-6 subjects per cohort. Within each new cohort there is no required waiting period between subjects. Subsequent cohorts are not opened until the first subject at the current dose level is observed for 21 days and subsequent subjects are observed for 14 days. More preferably, the starting dose is maintained unless toxicity or any other adverse indication requires reduction in dose.

Following surgery, up to three additional cycles of antibody-hormone therapy combination are administered, preferably within 45 days of prostatectomy. After surgery, antibody (IV) and 7.5 mg leuprolide (IM) are co-administered every twenty-eight days without bicalutamide.

It is noteworthy that in single agent studies in melanoma, 1 out of three patients treated with a single dose of 6 mg/kg of ticilimumab had diarrhea grade 3, and none of the patients treated with multiple doses of 6 mg/kg had diarrhea. In contrast, in the present study, 2 out of 3 patients treated with the combination of hormonal therapy and CTLA4 blockade had grade 3 diarrhea. Most notably, the severity (duration and magnitude) of the diarrhea was significantly higher than observed previously in any other study at any dose level. For instance, a patient received 3 mg/kg of ticilimumab and hormonal therapy exhibited prolonged diarrhea. This suggests an interaction between hormonal therapy and ticilimumab, such that the ability of ticilimumab to induce diarrhea was enhance. Diarrhea is considered to be pharmacologically mediated in that it is likely related to the mechanism of action of ticilimumab. Therefore, these data suggest that there is a biological interaction between HT and CTLA4 blockade, assuming that diarrhea is an immune-mediated effect. Thus, these data suggest a possible association between potential immune-mediated effects mediated by hormonal therapy and CTLA4 blockade. A possible correlation between tumor response in melanoma and putative “autoimmune breakthrough events” has been reported for single agent CTLA4 blockade in the absence of any other therapy (see Attia et al., J Clin Oncol. 23(25):6043-6053 (2005)). Thus, there may be a synergistic effect mediated by hormonal therapy and antibody therapy suggested by the data of increased severity of diarrhea observed in the present study.

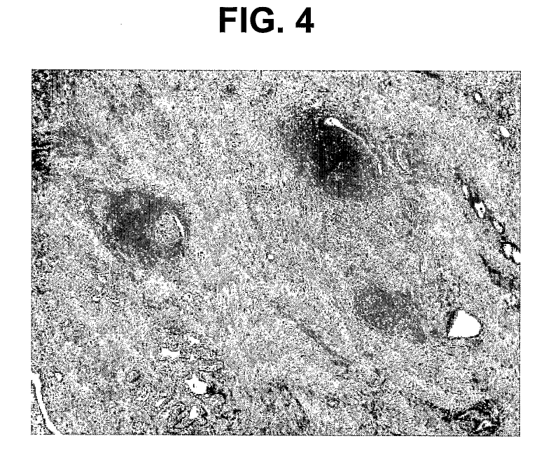

One cohort of three patients was enrolled at 6 mg/kg of ticilimumab (also known as antibody 11.2.1 or CP-675,206). One of the 3 patients (patient number 1005-1004) remained on study and underwent prostatectomy. The prostatectomy specimen showed detectable tumor regression and signs of an inflammatory infiltrate around tumor cells (FIG. 4). That is, after three months of antibody therapy and hormonal therapy (leuprolide and bicalutamide), extensive treatment effects and lymphocytic infiltrates were detected in a prostatectomy specimen obtained from a patient. The two other patients experienced diarrhea and did not complete the study.

Evidence of treatment effect was observed in the prostatectomy specimens from several patients treated with 1 mg/kg and 6 mg/kg. These effects included regression of carcinoma and benign prostate glandular tissue, lymphocytic infiltrates (see FIG. 4), and signs of acute and chronic inflammation. Both the magnitude of the observed changes in the glandular tissue, as well as the inflammatory and immune changes, cannot be solely attributed to the androgen blockade, and therefore suggest an immune-mediated effect of anti-CTLA4 antibody 11.2.1 (also known as ticilimumab) on the prostate glandular tissue.

### Example 2

### Anti-CTLA4 in Combination with at Least Two Hormonal Therapy Agents in the Adjuvant Treatment of Clinically Localized Prostate Cancer

Patients with clinically localized prostate cancer are administered (prior and during radiotherapy) both goserelin (ZOLADEX) and flutamide (EULEXIN) per hormonal therapy protocol, e.g., EULEXIN is administered 250 mg tid (three times a day), and ZOLADEX is administered 3.6 mg subcutaneously monthly for a total of four months (2 months prior and 2 months during radiotherapy). Alternatively, the patient receives 24 additional months of treatment with ZOLADEX. Such regimen for adjuvant therapy, typically administered with radiotherapy, but not with or after surgery, is described in, e.g., Hanks et al. J Clin Oncology 21: 3972-3978 (2003).

The patient is further administered a single IV infusion (100 mL/hr) of ticilimumab as described herein at a dose of about 3 mg/kg, or 6 mg/kg or 10 mg/kg or 15 mg/kg. Prophylactic anti-diarrheals are given as appropriate. The treatment is repeated after 28 days with anti-CTLA4 at the initial dose received, preferably, without dose escalation, every 28 days thereafter for maximum of 12 cycles in the absence of intolerable toxicity or disease progression. Alternatively, the antibody is administered every three months. ZOLADEX is co-administered with the antibody and/or every twenty-eight days, subcutaneously at 3.6 mg.

Preferably, the patient is premedicated with antihistamine (H1) at least one half hour prior to infusion of ticilimumab. Premedication is recommended but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 3

### Anti-CTLA4 in Combination with at Least Two Hormonal Therapy Agents in the Treatment of Patients with Rising PSA Prostate Cancer

Patients with prostate cancer and rising PSA following surgery or radiotherapy are administered either leuprolide (LUPRON) or goserelin (ZOLADEX), with or without bicalutamide (CASODEX) or flutamide (EULEXIN) per standard hormonal therapy protocol, e.g., LUPRON is administered 7.5 mg intramuscularly approximately every four weeks, ZOLADEX is administered 3.6 mg subcutaneously approximately every four weeks, CASODEX is administered 50 mg daily for fourteen days of the first cycle only, and EULEXIN is administered 250 mg tid daily. The patient is further administered a single IV infusion (100 mL/hr) of ticilimumab as described herein at a dose of about 1 mg/kg, 3 mg/kg, 6 mg/kg, 10 mg/kg or 15 mg/kg. Prophylactic anti-diarrheals are given as appropriate.

The treatment is repeated every 28 days thereafter without dose escalation, in the absence of intolerable toxicity or disease progression. LUPRON is administered every twenty-eight days, IM at 7.5 mg.

Preferably, the patient is premedicated with antihistamine (H1) at least one half hour prior to infusion of anti-CTLA4. Premedication is recommended but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 4

### Anti-CTLA4 in Combination with at Least One Hormonal Therapy Agent in the First-Line or Second-Line Treatment of Hormone-Independent Metastatic Prostate Cancer

Patients with metastatic prostate cancer (following surgery or radiotherapy) are administered both leuprolide (LUPRON) and bicalutamide (CASODEX) per standard hormonal therapy protocol, e.g., CASODEX is administered 50 mg daily for fourteen days of the first cycle only, and LUPRON is administered 7.5 mg intramuscularly approximately every four weeks. The patient is further administered a single IV infusion (100 mL/hr) of anti-CTLA4 antibodies as described herein at a dose of about 1 mg/kg, 3 mg/kg, 6 mg/kg, 10 mg/kg or 15 mg/kg. Prophylactic anti-emetics and anti-diarrheals are given as appropriate.

The treatment is repeated every 28 days thereafter without dose escalation, in the absence of intolerable toxicity or disease progression. LUPRON administered every twenty-eight days, IM at 7.5 mg.

Preferably, the patient is premedicated with antihistamine (H1) at least one half hour prior to infusion of anti-CTLA4. Premedication is recommended but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 5

### Anti-CTLA4 in Combination with at Least One Hormonal Therapy Agent in the First-Line or Second-Line Treatment of Hormone-independent Metastatic Prostate Cancer

Patients with metastatic prostate cancer (following surgery or radiotherapy) are administered both leuprolide (LUPRON) and bicalutamide (CASODEX) per standard hormonal therapy protocol, e.g., CASODEX is administered 50 mg daily for fourteen days of the first cycle only, and LUPRON is administered 7.5 mg intramuscularly approximately every four weeks. The patient is further administered a single IV infusion (100 mL/hr) of anti-CTLA4 antibody as described herein at a dose of about 15 mg/kg. Prophylactic anti-emetics and anti-diarrheals are given as appropriate.

The antibody is administered every three months thereafter without dose escalation, in the absence of intolerable toxicity or disease progression. Leuprolide administered every twenty-eight days, IM at 7.5 mg.

Preferably, the patient is premedicated with antihistamine (H1) at least one half hour prior to infusion of anti-CTLA4. Premedication is recommended but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 6

### Anti-CTLA4 in Sequential Combination with Hormonals in the Treatment of Hormone-Dependent Prostate Cancer

Patients with hormone-dependent prostate cancer, including patients who have not received prior hormonal therapy, are administered at least one course of hormone therapy comprising at least one hormone therapy agent per standard hormonal therapy protocols (e.g., leuprolide (LUPRON) is administered 7.5 mg intramuscularly approximately every four weeks, goserelin (ZOLADEX) is administered 3.6 mg subcutaneously approximately every four weeks, bicalutamide (CASODEX) is administered 50 mg daily for fourteen days of the first cycle only, and flutamide (EULEXIN) is administered 250 mg tid daily, among others). Following at least one course of hormone therapy and after allowing at least one week but less than four months to pass after administration of the last dose of hormonal therapy agent, the patient is sequentially administered a single IV infusion (100 mL/hr) of ticilimumab as described herein at a dose of at least 10 mg/kg every four weeks. Prophylactic anti-diarrheals are given as appropriate.

The treatment is repeated as indicated with or without dose escalation, in the absence of intolerable toxicity or disease progression.

The patient is premedicated with antihistamine (H1) at least one half hour prior to infusion of anti-CTLA4, but premedication is not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 7

### Anti-CTLA4 in Sequential Combination with Hormonals in the Treatment of Hormone-Dependent Prostate Cancer

Patients with hormone-dependent prostate cancer, including patients who have not received prior hormonal therapy, are administered at least one course of hormone therapy comprising at least one hormone therapy agent per standard hormonal therapy protocols (e.g., leuprolide (LUPRON) is administered 7.5 mg intramuscularly approximately every four weeks, goserelin (ZOLADEX) is administered 3.6 mg subcutaneously approximately every four weeks, bicalutamide (CASODEX) is administered 50 mg daily for fourteen days of the first cycle only, and flutamide (EULEXIN) is administered 250 mg tid daily, among others). Following at least one course of hormone therapy and after allowing at least one week but less than four months to pass after administration of the last dose of hormonal therapy agent, the patient is sequentially administered a single IV infusion (100 mL/hr) of ticilimumab as described herein at a dose of at least 15 mg/kg about every three months. Prophylactic anti-diarrheals are given as appropriate.

The treatment is repeated as indicated with or without dose escalation, in the absence of intolerable toxicity or disease progression.

The patient is premedicated with antihistamine (H1) at least one half hour prior to infusion of anti-CTLA4, but premedication is not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 8

### Anti-CTLA4 in Sequential Combination with at Least One Hormonal Therapy Agent in the Treatment of Hormone-Independent Prostate Cancer

Patients with hormone-independent prostate cancer, e.g., following previous hormonal therapy (e.g., leuprolide (LUPRON), goserelin (ZOLADEX), bicalutamide (CASODEX), flutamide (EULEXIN), and combinations thereof per standard hormonal therapy protocols), are administered at least one course of hormone therapy comprising at least one hormone therapy agent per standard hormonal therapy protocols (e.g., LUPRON is administered 7.5 mg intramuscularly approximately every four weeks, ZOLADEX is administered 3.6 mg subcutaneously approximately every four weeks, CASODEX is administered 50 mg daily for fourteen days of the first cycle only, and EULEXIN is administered 250 mg tid daily). Following at least one course of hormone therapy, and after allowing a period of at least one week but not more than about four months after the last dose of hormonal therapy agent is administered, the patient is administered a single IV infusion (100 mL/hr) of ticilimumab as described herein at a dose of at least 10 mg/kg every twenty eight days.

The treatment is repeated without dose escalation, in the absence of intolerable toxicity or disease progression. For instance, LUPRON is administered IM every twenty-eight days at 7.5 mg.

The patient is premedicated with antihistamine (H1) at least one half hour prior to infusion of ticilimumab, but premedication is but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

### Example 9

### Anti-CTLA4 in Sequential Combination with at Least One Hormonal Therapy Agent in the Treatment of Hormone-Independent Prostate Cancer

Patients with hormone-independent prostate cancer, e.g., following previous hormonal therapy (e.g., leuprolide (LUPRON), goserelin (ZOLADEX), bicalutamide (CASODEX), flutamide (EULEXIN), and combinations thereof per standard hormonal therapy protocols), are administered at least one course of hormone therapy comprising at least one hormone therapy agent per standard hormonal therapy protocols (e.g., LUPRON is administered 7.5 mg intramuscularly approximately every four weeks, ZOLADEX is administered 3.6 mg subcutaneously approximately every four weeks, CASODEX is administered 50 mg daily for fourteen days of the first cycle only, and EULEXIN is administered 250 mg tid daily). Following at least one course of hormone therapy, and after allowing a period of at least one week but not more than about four months after the last dose of hormonal therapy agent is administered, the patient is administered a single IV infusion (100 mL/hr) of ticilimumab as described herein at a dose of at least 15 mg/kg every three months.

The treatment is repeated without dose escalation, in the absence of intolerable toxicity or disease progression. For instance, LUPRON is administered IM every twenty-eight days at 7.5 mg.

The patient is premedicated with antihistamine (H1) at least one half hour prior to infusion of ticilimumab, but premedication is but not required.

Also, an agent with anti-diarrheal effect may be administered, including those agents indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), and PSA level, are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. The antibody has the heavy and light chain amino acid sequences of ticilimumab. The anti-CTLA4 antibody is ticilimumab.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for the treatment of prostate cancer in a patient in need of such treatment, said method comprising administering to said patient a) an amount of a hormonal therapy agent and b) an amount of an antibody, or antigen-binding portion thereof, that binds human CTLA4, wherein said antibody or portion is first administered more than one day and less than twenty-eight days following administration of said hormonal therapy agent, and wherein said amounts are effective in combination for said treatment.

2. The method of claim 1, wherein said antibody or portion thereof is administered more than two days following administration of said hormonal therapy agent.

3. The method of claim 1, wherein said antibody or portion thereof is administered less than twenty-one days following administration of said hormonal therapy agent.

4. The method of claim 1, wherein administration of said hormonal therapy agent terminates prior to said first administration of said antibody or portion thereof.

5. The method of claim 1, wherein said hormonal therapy agent is selected from the group consisting of an anti-androgen, a gonadotropin-releasing hormone (GnRH) antagonist, and a luteinizing hormone-releasing hormone (LH-RH) agonist.

6. The method of claim 1, wherein said cancer is selected from hormone-dependent cancer and hormone-independent cancer.

7. The method of claim 1, wherein said cancer is hormone-independent and said administration of hormonal therapy terminates prior to said first administration of said antibody or portion thereof.

8. The method of claim 1, wherein said antibody is administered according to a regimen selected from administering about 10 mg/kg every twenty-eight days and administering about 15 mg/kg every three months.

9. The method of claim 1, wherein said anti-CTLA4 antibody, or antigen-binding portion thereof, is at least one antibody selected from the group consisting of:
(a) a human antibody having a binding affinity for CTLA4 of about 10⁻⁸ or greater, and which inhibits binding between CTLA4 and B7-1, and binding between CTLA4 and B7-2;
(b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab;
(c) a human antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, and 12.9.1.1;
(d) a human antibody having the amino acid sequences of a heavy chain variable region and a light chain variable region of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab;
(e) an antibody, or antigen-binding portion thereof, that competes for binding with CTLA4 with at least one antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab; and
(f) an antibody, or antigen-binding portion thereof, that cross-competes for binding with CTLA4 with at least one antibody having the heavy and light chain amino acid sequences of an antibody selected from the group consisting of 4.1.1, 4.8.1, 4.10.2, 4.13.1, 4.14.3, 6.1.1, ticilimumab, 11.6.1, 11.7.1., 12.3.1.1, 12.9.1.1, and ipilimumab.

10. The method of claim 1, wherein said antibody is a human antibody having the heavy and light chain amino acid sequences of ticilimumab.

11. The method of claim 1, wherein said antibody comprises a heavy chain and a light chain wherein the amino acid sequences of the heavy chain variable region of said heavy chain and the light chain variable region of said light chain are selected from the group consisting of:
(a) the amino acid sequence of SEQ ID NO:3 and the amino acid sequence of SEQ ID NO:9;
(b) the amino acid sequence of SEQ ID NO:15 and the amino acid sequence of SEQ ID NO:21;
(c) the amino acid sequence of SEQ ID NO:27 and the amino acid sequence of SEQ ID NO:33;
(d) the amino acid sequence encoded by the nucleic acid sequence of SEQ ID NO:1 and the amino acid sequence encoded by the nucleic acid sequence of SEQ ID NO:7;
(e) the amino acid sequence encoded by the nucleic acid sequence of SEQ ID NO:13 and the amino acid sequence encoded by the nucleic acid sequence of SEQ ID NO:19;
(f) the amino acid sequence encoded by the nucleic acid sequence of SEQ ID NO:25 and the amino acid sequence encoded by the nucleic acid sequence of SEQ ID NO:31;
(g) the amino acid sequence of a heavy chain variable region and a light chain variable region of ipilimumab.

12. The method of claim 1, wherein said antibody, or antigen-binding portion thereof, is an antibody selected from the group consisting of:
(a) an antibody having a heavy chain variable region comprising the amino acid sequences set forth in SEQ ID NO:4, SEQ ID NO:5, and SEQ ID NO:6, and further having a light chain variable region comprising the amino acid sequences set forth in SEQ ID NO:10, SEQ ID NO:11 and SEQ ID NO:12;
(b) an antibody having a heavy chain variable region comprising the amino acid sequences set forth in SEQ ID NO:16, SEQ ID NO:17, and SEQ ID NO:18, and further having a light chain variable region comprising the amino acid sequences set forth in SEQ ID NO:22, SEQ ID NO:23 and SEQ ID NO:24;
(c) an antibody having a heavy chain variable region comprising the amino acid sequences set forth in SEQ ID NO:28, SEQ ID NO:29, and SEQ ID NO:30, and further having a light chain variable region comprising the amino acid sequences set forth in SEQ ID NO:34, SEQ ID NO:35 and SEQ ID NO:36; and
(d) an antibody having a heavy chain variable region comprising the amino acid sequences of the heavy chain CDR1, CDR2, and CDR3 of antibody ipilimumab, further having a light chain variable region comprising the amino acid sequences of the light chain CDR1, CDR2, and CDR3 of antibody ipilimumab.

13. A method for the treatment of a hormone-independent prostate cancer in a patient in need of such treatment, said method comprising administering to said patient an amount of an antibody that binds human CTLA4, or antigen-binding portion thereof, and an amount of a hormonal therapy agent, wherein said hormonal therapy agent is administered in multiple doses for a time greater than one month and wherein said antibody, or portion thereof, is administered during the period of administration of said hormonal therapy agent, and wherein said amounts are effective in combination for said treatment.

14. The method of claim 13, wherein said hormonal therapy agent is administered over a period greater than two months.

15. The method of claim 14, said method further comprising administering multiple doses of said antibody, or portion thereof, over a period greater than one month that overlaps with said period of administration of said hormonal therapy agent.

16. The method of claim 15, wherein said period of administration of said antibody, or portion thereof, and said period of administration of said hormonal therapy agent overlap by more than two months.

17. The method of claim 15, wherein said multiple doses of said antibody, or portion thereof, and said period of administration of said hormonal therapy agent overlap by more than six months.

18. A method for the treatment of hormone-dependent prostate cancer in a patient in need of such treatment, said method comprising co-administering to said patient a therapeutically effective amount of an anti-CTLA4 antibody, or antigen-binding portion thereof, and a therapeutically effective amount of at least two hormonal therapy agents, wherein said agent is selected from the group consisting of an anti-androgen, a gonadotropin-releasing hormone (GnRH) antagonist, and a luteinizing hormone-releasing hormone (LH-RH) agonist.

19. The method of claim 18, wherein said anti-androgen is bicalutamide and said agonist is leuprolide.

20. A pharmaceutical composition for treatment of prostate cancer, said composition comprising a therapeutically effective amount of an anti-CTLA4 antibody, or antigen-binding portion thereof, and a therapeutically effective amount of at least two hormonal therapy agents, wherein said hormonal therapy agent is selected from the group consisting of an anti-androgen, a gonadotropin-releasing hormone (GnRH) antagonist, and a luteinizing hormone-releasing hormone (LH-RH) agonist.
